(19) United States
(12) Patent Application Publication
(10) Pub. No.: US 2006/0019333 A1
(43) Pub. Date: Jan. 26, 2006

(54) Control of reactor environmental conditions

(76) Inventors: Seth T. Rodgers, Somerville, MA (US); A. Peter Russo, Woburn, MA (US); Sean J. LeBlanc, Westminster, MA (US); Denis F. Leroux, Medford, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 11/146,920

(22) Filed: Jun. 7, 2005

Related U.S. Application Data
(60) Provisional application No. 60/577,985, filed on Jun. 7, 2004.

(51) Int. Cl. C12P 1/00

(52) U.S. Cl. 435/41

(57) ABSTRACT

The present disclosure generally relates to chemical, biological, and/or biochemical reactor chips and/or reaction systems such as microreactor systems, as well as methods for constructing and using such systems. In some cases, humidity control materials are utilized to provide beneficially high rates of gas exchange. The humidity control materials may be used, in certain instances, to provide at least adequate, and in certain embodiments superior, gas exchange for systems having small volumes. In some cases, the currently disclosed materials include certain polymers, e.g., poly(acetylene)s such as poly(alkylacetylene)s. The polymers may be at least partially halogenated (for example, fluorinated) in some instances. In certain embodiments, a chip and/or a reaction system may be constructed so as to promote cell growth within it. In some embodiments, the chips may include one or more reaction sites. The reaction sites can be very small, for example, with a volume of less than about 1 ml. In certain embodiments, a reaction system is able to detect, measure and/or control an environmental factor such as the temperature, pressure, CO₂ concentration, O₂ concentration, relative humidity, pH, etc., associated with one or more reaction sites, by using one or more sensors, actuators, processors, and/or control systems. In certain embodiments, the present disclosure discloses materials and systems having humidity and/or gas control, for example, for use with a reaction system. Such materials may have high oxygen permeability and/or low water vapor permeability. In certain embodiments, the disclosed devices can employ light-interacting components suitable for use in reaction systems. These components may include waveguides, optical fibers, light sources, photodetectors, optical elements, and the like.

### RELATED APPLICATIONS

This application claims the benefit of U.S. Provisional Patent Application Ser. No. 60/577,985, filed Jun. 7, 2004, entitled “Control of Reactor Environmental Conditions,” by Rodgers, et al., incorporated herein by reference.

### FIELD OF THE INVENTION

The present invention generally relates to chemical, biological, and/or biochemical reactor chips and/or reaction systems such as microreactor systems, and, in particular, to chemical, biological, and/or biochemical reactor chips having humidity control materials.

### BACKGROUND

A wide variety of reaction systems are known for the production of products of chemical and/or biochemical reactions. Chemical plants involving catalysis, biochemical fermenters, pharmaceutical production plants, and a host of other systems are well-known. Biochemical processing may involve the use of a live microorganism (e.g., cells) to produce a substance of interest.

Cells are cultured for a variety of reasons. Increasingly, cells are cultured for the proteins or other valuable materials they produce. Many cells require specific conditions, such as a controlled environment. The presence of nutrients, metabolic gases such as oxygen and/or carbon dioxide, humidity, as well as other factors such as temperature, may affect cell growth. Cells require time to grow, during which favorable conditions must be maintained. In some cases, such as with particular bacterial cells, a successful cell culture may be performed in as little as 24 hours. In other cases, such as with particular mammalian cells, a successful culture may require about 30 days or more.

Typically, cell cultures are performed in media suitable for cell growth and containing necessary nutrients. The cells are generally cultured in a location, such as an incubator, where the environmental conditions can be controlled. Incubators traditionally range in size from small incubators (e.g., about 1 cubic foot) for a few cultures up to an entire room or rooms where the desired environmental conditions can be carefully maintained.

Recently, as described in International Patent Application No. PCT/US01/07679, filed Mar. 9, 2001, entitled “Microreactor,” by Jury, et al., published as WO 01/68257 on Sep. 20, 2001 incorporated herein by reference, cells have also been cultured on a very small scale (i.e., on the order of a few milliliters or less), so that, among other things, many cultures can be performed in parallel. As is appreciated in the art, providing and maintaining suitable culture conditions for cell viability and growth in such small-scale systems can be extremely difficult. Typical materials utilized for fabrication of such small-scale bioreactors often are not ideally suited for establishing and maintaining desirable culture conditions because of, for example, low permeability to nutrient gases (e.g. O₂ and/or CO₂), high permeability to water vapor leading to loss of culture medium liquid volume and a concomitant deleterious change in osmolality and dissolved substance concentration, etc.

### SUMMARY OF THE INVENTION

The present disclosure includes description of chemical, biological, and/or biochemical reactor chips and/or reaction systems such as microreactor systems having humidity control materials. In some cases, the humidity control materials provide beneficially high rates of gas exchange for selected gases. The humidity control materials may be used, in certain instances, to provide at least adequate gas exchange for systems having small volumes. The subject matter of this invention involves, in some cases, interrelated products, alternative solutions to a particular problem, and/or a plurality of different uses of one or more systems and/or articles.

In one set of embodiments, an apparatus is disclosed that includes a substrate comprising a predetermined reaction site, and a material positioned adjacent the predetermined reaction site. In one embodiment, the material comprises a polymer including a structure: 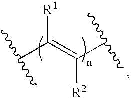
where n is at least 1, and each of R¹ and R² independently comprises an atom. In certain cases, simultaneously, R¹ is not methyl and R² is not trimethylsilyl.

In another embodiment, the material comprises a polymer including a structure: 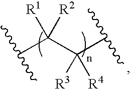
where n is at least 1, and each of R¹, R², R³, and R⁴ independently comprises an atom. In certain cases, simultaneously, R¹ is not H, R² is not H, R³ is not H, and R⁴ is neither H nor has a structure: 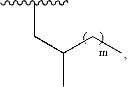
where m is an integer between 0 and 3 (inclusively).

In yet another embodiment, the material comprises a polymer including a structure: 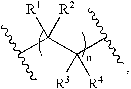
where n is at least 1, and each of R¹, R², R³, and R⁴ independently is hydrogen, a halogen, or a carbon-containing moiety. In certain instances, at least one of R¹, R², R³, or R⁴ is a carbon-containing moiety that comprises a halogen, for example, fluorine.

In still another embodiment, the material comprises a polymer including a structure: 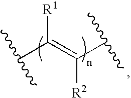
where n is at least 1, and each of R¹ and R² independently comprises an atom. In certain instances, at least one of R¹ or R² is a carbon-containing moiety that comprises a halogen, for example, fluorine.

In some cases, the predetermined reaction site can have a volume of less than about 1 ml. In particular instances, the substrate may be constructed and arranged to maintain at least one living cell at the predetermined reaction site. In certain embodiments, the material positioned adjacent the predetermined reaction site includes a humidity control material.

In another set of embodiments, the a series of methods are disclosed. In one set of embodiments, the method includes an act of culturing at least one living cell proximate a material comprising a polymer including a structure: 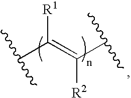
where n is at least 1, and each of R¹ and R² independently comprises an atom. In certain cases, simultaneously, R¹ is not methyl and R² is not trimethylsilyl.

In another set of embodiments, the method includes an act of culturing at least one living cell proximate a material comprising a polymer including a structure: 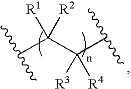
where n is at least 1, and each of R¹, R², R³, and R⁴ independently comprises an atom. In some cases, simultaneously, R¹ is not H, R² is not H, R³ is not H, and R⁴ is neither H nor has a structure: 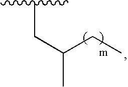
m being an integer between 0 and 3 (inclusively).

In yet another set of embodiments, the method includes an act of culturing at least one living cell proximate a material comprising a polymer including a structure: 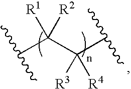
where n is at least 1, and each of R¹, R², R³, and R⁴ independently is hydrogen, a halogen, or a carbon-containing moiety. In certain instances, at least one of R¹, R², R³, or R⁴ is a carbon-containing moiety that comprises a halogen, for example, fluorine.

In still another set of embodiments, the method includes an act of culturing at least one living cell proximate a material comprising a polymer including a structure: 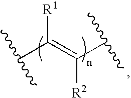
where n is at least 1, and each of R¹ and R² independently comprises an atom. In certain instances, at least one of R¹ or R² is a carbon-containing moiety that comprises a halogen, for example, fluorine.

In some cases, the predetermined reaction site can have a volume of less than about 1 ml. In particular instances, the substrate may be constructed and arranged to maintain at least one living cell at the predetermined reaction site. In certain embodiments, the material positioned adjacent the predetermined reaction site includes a humidity control material.

Also disclosed are methods of making a chip and/or a reaction system, e.g., as described in any of the embodiments herein. Also disclosed are methods of using a chip and/or a reaction system, e.g., as described in any of the embodiments herein. Also disclosed are methods of promoting, fabricating, using, and/or selling of a chip and/or a reaction system, e.g., as described in any of the embodiments herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two (or more) applications incorporated by reference include conflicting and/or inconsistent disclosure with respect to each other, then the later-filed application shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIG. 1 is a schematic illustration of a portion of a microfluidic chip comprising a plurality of reaction sites according to one embodiment of the invention;

FIG. 2 illustrates an example of a microfluidic chip for use with the invention including mixing, heating/dispersion, reaction, and separation units, in expanded view;

FIGS. 3A-3C illustrate various stackable arrangements of chips of the invention;

FIGS. 4A and 4B illustrate a chip device having multiple layers and comprising a plurality of reaction sites according to one embodiment of the invention;

FIG. 5 is a block diagram of an example of a control system of the invention;

FIGS. 6A and 6B are cross sectional views of certain embodiments of the present invention;

FIGS. 7A-7D illustrates certain membranes of the invention in fluid communication with various reaction sites.

FIGS. 8A and 8B (expanded) illustrate portions of various chips according to one embodiment of the invention;

FIGS. 9A and 9B illustrate expanded views of portions of various chips according to another embodiment of the invention; and

FIG. 10 illustrates an expanded view of a portion of a chip according to yet another embodiment of the invention.

### DETAILED DESCRIPTION

The present disclosure generally relates to chemical, biological, and/or biochemical reactor chips and/or reaction systems such as microreactor systems, as well as methods for constructing and using such systems. In some cases, humidity control materials are utilized to provide beneficially high rates of gas exchange. The humidity control materials may be used, in certain instances, to provide at least adequate, and in certain embodiments superior, gas exchange for systems having small volumes. In some cases, the currently disclosed materials include certain polymers, e.g., poly(acetylene)s such as poly(alkylacetylene)s. The polymers may be at least partially halogenated (for example, fluorinated) in some instances. In certain embodiments, a chip and/or a reaction system may be constructed so as to promote cell growth within it. In some embodiments, the chips may include one or more reaction sites. The reaction sites can be very small, for example, with a volume of less than about 1 ml. In certain embodiments, a reaction system is able to detect, measure and/or control an environmental factor such as the temperature, pressure, CO₂ concentration, O₂ concentration, relative humidity, pH, etc., associated with one or more reaction sites, by using one or more sensors, actuators, processors, and/or control systems. In certain embodiments, the present disclosure discloses materials and systems having humidity and/or gas control, for example, for use with a reaction system. Such materials may have high oxygen permeability and/or low water vapor permeability. In certain embodiments, the disclosed devices can employ light-interacting components suitable for use in reaction systems. These components may include waveguides, optical fibers, light sources, photodetectors, optical elements, and the like.

Each of the following commonly-owned applications directed to related subject matter and/or disclosing methods and/or devices and/or materials useful or potentially useful for the practice of the present invention is incorporated herein by reference: U.S. patent application Ser. No. 09/707,852, filed Nov. 7, 2000, entitled “Microreactor,” by Jury, et al.; International Patent Application No. PCT/US01/07679, filed Mar. 9, 2001, entitled “Microreactor,” by Jury, et al., published as WO 01/68257 on Sep. 20, 2001; U.S. patent application Ser. No. 10/119,917, filed Apr. 10, 2002, entitled “Microfermentor Device and Cell Based Screening Method,” by Zarur, et al., published as 2003/0077817 on Apr. 24, 2003; U.S. patent application Ser. No. 10/457,049, filed Jun. 5, 2003, entitled “Materials and Reactor Systems having Humidity and Gas Control,” by Rodgers, et al,. published as 2004/0058437 on Mar. 25, 2004; U.S. patent application Ser. No. 10/457,015, filed Jun. 5, 2003, entitled “Reactor Systems Having a Light-Interacting Component,” by Miller, et al., published as 2004/0058407 on Mar. 25, 2004; U.S. patent application Ser. No. 10/664,046, filed Sep. 16, 2003, entitled “Determination and/or Control of Reactor Environmental Conditions,” by Miller, et al., published as 2004/0132166 on Jul. 8, 2004; U.S. patent application Ser. No. 10/664,068, filed Sep. 16, 2003, entitled “Systems and Methods for Control of pH and Other Reactor Environmental Conditions,” by Miller, et al., published as 2005/0026134 on Feb. 3, 2005; U.S. patent application Ser. No. 10/664,067, filed Sep. 16, 2003, entitled “Microreactor Architecture and Methods,” by Rodgers, et al., published as 2005/0032204 on Feb. 10, 2005; U.S. Patent Application Ser. No. 60/577,985, filed Jun. 7, 2004, entitled “Control of Reactor Environmental Conditions,” by Rodgers, et al.; U.S. Patent Application Serial No. 10/863,585, filed Jun. 7, 2004, entitled “System and Method for Process Automation,” by Rodgers, et al., published as 2005/0037485 on Feb. 17, 2005; U.S. patent application Ser. No. 10/863,584, filed Jun. 7, 2004, entitled “Apparatus and Method for Manipulating Substrates,” by Zarur, et al., published as 2005/0019904 on Jan. 27, 2005;U.S. patent application Ser. No. 10/863,636, filed Jun. 7, 2004, entitled “Reactor with Memory Component,” by Zarur, et al., published as 2005/0026273 on Feb. 3, 2005; U.S. Patent Application Ser. No. 60/577,977, filed Jun. 7, 2004, entitled “Gas Control in a Reactor,” by Rodgers, et al.; U.S. Patent Application Ser. No. 60/577,986, filed Jun. 7, 2004, entitled “Reactor Mixing” by Johnson, et al.; U.S. patent application Ser. No. 10/927,789, filed Aug. 27, 2004, entitled “Rotatable Reactor Systems and Methods,” by Zarur, et al.; and U.S. Patent Application Ser. No. 60/609,721, filed Sep. 14, 2004, entitled “Inlet Channel Volume in a Reactor,” by Miller, et al.

In certain embodiments, the present disclosure is directed to a device, such as a chip, able to control gases or humidity therein. In some embodiments, the disclosed devices may allow humidity control to be passive and built into a chip or other device that may be used to, for example, conduct chemical or biochemical reactions, and/or culture cells. In one embodiment, humidity control or maintenance may be provided to the device in the form of a humidity controller and/or a film, optionally with low water permeability relative to the oxygen permeability. As used herein, a “humidity controller” is a device that allows certain gases, such as oxygen, carbon dioxide, and/or nitrogen to enter the chip (or other device), but inhibits the passage of water vapor into the chip (or other device). The humidity controller may allow passage of small amounts of water vapor into the chip (or other device), but does not allow as much water vapor to enter the chip as at least one other gas, e.g. one or more of those listed above. Examples include, but are not limited to, membranes and thin films (e.g., films having a thickness of less than 2 mm). In some embodiments, the humidity controller may be positioned as, or in, a wall of a chip or other device, such as within a wall of a reactor unit or reaction site. In other embodiments, the humidity controller may be positioned such that it is in fluid communication with one or more reaction sites. In some embodiments, each of the reaction sites in the chip may be adjacent to, and/or in fluid communication with a humidity controller.

Humidity controllers disclosed herein can include a humidity control material designed to maximize gas and/or minimize water vapor passage therethrough. The humidity control material may allow the passage of certain desired gases, such as oxygen and/or carbon dioxide, while inhibiting the passage of other gases, for example, water vapor. The presently disclosed humidity control materials may be suitable for use as a humidity controller in a chip, reaction system, or other device, but are not limited to such uses; rather, the materials may be used anywhere where water vapor or other specified gases are to be kept in or out, while allowing the passage of oxygen and/or other gases. For example, the disclosed humidity control materials may be useful in greenhouses or wound dressings.

In one set of embodiments, the humidity control material includes a polymer. The polymer may include, for instance, a poly(acetylene) and/or a poly(alkylacetylene) such as poly(2-alkylacetylene). Examples of poly(2-alkylacetylene)s include, but are not limited to, poly(2-hexyne), poly(2-heptyne), poly(2-octyne), poly(2-nonyne), poly(2-decyne), poly(2-undecyne), etc. In one embodiment, the poly(acetylene) comprises a structure: 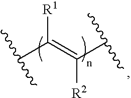
where n is at least 1. Each of R¹ and R² may independently comprise any atom and/or functional group, for example, hydrogen, a halogen or a pseudohalogen, an alkyl, an aryl, an alkylaryl, an arylalkyl, a cyclic group, a hydroxide, an alcohol, a thiol, a carboxylic acid, a silyl, etc. Some of these are further described below. For example, R¹ and/or R² may each independently be hydrogen or a straight-chain alkyl, such as propyl, pentyl, hexyl, heptyl, octyl, nonyl, decyl, undecyl, etc. In certain cases, the poly(acetylene) is one other than poly(1-trimethylsilyl-1-propyne), i.e., simultaneously, R¹ is not methyl and R² is not trimethylsilyl. In certain instances, R¹ and R² are not both H.

In another embodiment, the polymer may include a structure: 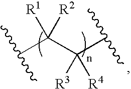
where n is at least 1. Each of R¹, R², R³, and R⁴ may be any atom and/or functional group, e.g., as described above. In one particular case, simultaneously, R¹ is not H, R² is not H, R³ is not H, and R⁴ is neither H nor has a structure: 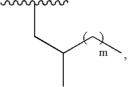
where m is an integer between 0 and 3, inclusive.

In some embodiments, the polymer is halogenated (i.e., comprises at least one halogen atom), for example, fluorine. Non-limiting examples include a fluorinated poly(acetylene), a fluorinated poly(alkylacetylene), a chlorinated poly(acetylene), a chlorinated poly(alkylacetylene), a fluorinated and chlorinated poly(acetylene), a fluorinated and chlorinated poly(alkylacetylene), etc. Halogenated polymers may be useful, for example, to reduce water vapor permeability of the humidity control material, for instance, due to the increased hydrophobicity of the fluorinated polymer. Examples of poly(2-alkylacetylene)s that may include one or more halogen atoms include, but are not limited to, poly(2-hexyne), poly(2-heptyne), poly(2-octyne), poly(2-nonyne), poly(2-decyne), poly(2-undecyne), etc. In one embodiment, the polymer comprises a structure: 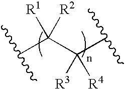
where n is at least 1, and at least one of R¹, R², R³, or R⁴ comprises a halogen (alone, or in association with other atoms, for example, a carbon-containing moiety), e.g., fluorine, chlorine, bromine, etc. Each of R¹, R², R³, and R⁴ may independently comprise any atom and/or functional group, for example, hydrogen, a halogen or a pseudohalogen, an alkyl, an aryl, an alkylaryl, an arylalkyl, a cyclic group, a hydroxide, an alcohol, a thiol, a carboxylic acid, a silyl, etc. In some cases, one or more of R¹, R², R³, or R⁴ may be saturated in halogens, e.g., fluorine atoms.

As one example, one of R¹, R², R³, and R⁴ may have a structure: 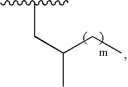
where m is an integer greater than or equal to 0, and one or more carbon atoms in this structure has one or more halogen atom bonded to it, i.e., if m is 0, then this structure would be: 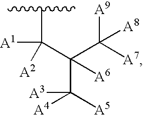
and at least one of A¹, A², A³, A⁴, A⁵, A⁶, A⁷, A⁸, or A⁹ is a halogen atom, e.g., fluorine, chlorine, bromine, etc.

In another embodiment, the polymer has a structure: 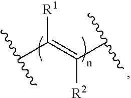
where n is at least 1. and each of R¹ and R² independently comprises an atom such that at least one of R¹ or R² is a halogen or a carbon-containing moiety that comprises a halogen atom (alone, or in association with other atoms, for example, a carbon-containing moiety), e.g., fluorine, chlorine, bromine, etc. R¹ and R² may independently comprise any atom and/or functional group, for example, hydrogen, a halogen or a pseudohalogen, an alkyl, an aryl, an alkylaryl, an arylalkyl, a cyclic group, a hydroxide, an alcohol, a thiol, a carboxylic acid, a silyl, etc. In some cases, one or more of R¹, R², R³, or R⁴ may be saturated in halogens, e.g., fluorine atoms.

In one embodiment, at least one of R¹ or R² comprises a silicon atom. For instance, the polymer may have a structure: 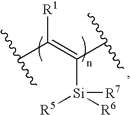
where at least one of R¹, R⁵, R⁶, or R⁷ comprises a halogen, e.g., fluorine, chlorine, bromine, etc. For instance R¹, R⁵, R⁶, or R⁷ may be halogen such as fluorine, be a carbon-containing moiety that comprises halogen such as a halogenated alkyl, etc.

Monomers useful for forming some of the above-described polymers are commercially available, for example, from GFS Chemicals, Inc. (Powell, Ohio), or Lancaster Synthesis, Inc. (Windham, N.H.). Suitable techniques for polymerizing the monomers are known to those of ordinary skill in the art, or involve no more than routine modifications of known techniques, and are described, for example, in Pinnau I., et al. “Influence of Side-Chain Length on the Gas Permeation Properties of Poly(2-alkylacetylenes), Macromolecules, 37(8): 2823-2828, 2004. For example, MoCl₅ and/or Ph₄Sn may used to catalyze certain polymerization reactions useful for producing the above-disclosed polymers.

The term “halogen,” or equivalently, “halogen atom,” as used herein, is given its ordinary meaning as used in the field of chemistry. The halogens are fluorine, chlorine, bromine, iodine, and astatine, and may have any charge state and/or electronic configuration. In some embodiments, the polymers include one or more halogen atoms comprising fluorine, chlorine, bromine, or iodine. In certain cases, the polymer includes fluorine, chlorine, and bromine; fluorine and chlorine; chlorine and bromine, or a single type of halogen atom.

As used herein, “alkyl” is given its ordinary meaning as used in the field of organic chemistry. Alkyl (i.e., aliphatic) moieties useful for practicing the invention can contain any of a wide number of carbon atoms, for example, between and 1 and 25 carbon atoms, between 1 and 20 carbon atoms, between 1 and 15 carbon atoms, between 1 and 10 carbon atoms, or between 1 and 5 carbon atoms. In some cases, the alkyl moiety will contain at least 1 carbon atom, at least 3 carbon atoms, at least 5 carbon atoms, or at least 10 carbon atoms; in other cases, the alkyl moiety will have at most 10 carbon atoms, at most 5 carbon atoms, or at most 3 carbon atoms.

The carbon atoms within the alkyl moiety may be arranged in any configuration within the alkyl moiety, for example, as a straight chain (i.e., a n-alkyl such as methyl, ethyl, propyl, butyl, pentyl, hexyl, heptyl, octyl, nonyl, decyl, undecyl, etc.) or a branched chain, i.e., a chain where there is at least one carbon atom that is covalently bonded to at least three carbon atoms (e.g., a t-butyl moiety, an isoalkyl moiety such as an isopropyl moiety or an isobutyl moiety, etc.). The alkyl moiety may contain only single bonds, or may contain one or more double and/or triple bonds within its structure, for example, as in an alkene, an alkyne, an alkadiene, an alkadiyne, an alkenyne, etc. In some cases, the alkyl moiety contains only carbon and hydrogen atoms; however, in other cases, the alkyl moiety may also contain one or more substituents, i.e., a non-carbon and non-hydrogen moiety may be present within the alkyl moiety. For example, in certain cases, the alkyl moiety can include a halogen, an alkoxy moiety (e.g., methoxy or ethoxy), an amine moiety (e.g., a primary, secondary, or tertiary amine), a carbonyl (e.g., an aldehyde and/or a ketone) or a hydroxide as a substituent. If more than substituent is present within the alkyl moiety, then the substituents may each be the same or different.

Similarly, a “cyclic” moiety, as used herein, is given its ordinary definition as used in the field of organic chemistry, i.e., a moiety that contains at least one ring of atoms, and may contain more than one ring of atoms. That is, a cyclic moiety has at least one chain of atoms that does not have a terminal end. The chain may have, for example, three, four, five, six, seven, eight, nine, or ten or more atoms arranged in a ring. In some cases, the cyclic moiety has a maximum size of at most ten atoms, at most eight atoms, or at most seven atoms. In some cases, the cyclic moiety may only include carbon and hydrogen atoms; however, in other cases, the atoms may also include, besides carbon atoms, nitrogen atoms, oxygen atoms, sulfur atoms, silicon atoms, or any other atom able to covalently bond to at least two different atoms (i.e., a “heterocyclic” moiety). If the cyclic moiety contains more than one ring, the rings may be arranged in any orientation with respect to each other, e.g., the rings may be fused (i.e., at least two rings have more than one atom in common, for example, as in bicyclic moieties, tricyclic moieties, etc.), spiro (i.e., two rings have only one atom in common), a ring may be a substituent on another ring, two or more rings may be connected through an alkyl moiety, etc.

The cyclic moiety may be a saturated cyclic moiety (i.e., a moiety not containing any double or triple bonds, such as a cyclopentyl moiety, a cyclohexyl moiety, a cycloheptyl moiety, a cyclooctyl moiety, etc.) or an unsaturated cyclic moiety (i.e., a moiety containing at least one double or triple bond, such as a cycloalkenyl moiety, a cycloalkynyl moiety, an aromatic moiety, etc.). An “aromatic” moiety is given its ordinary meaning as used in the art, i.e., a moiety having at least one ring in which some electrons are delocalized in the ring. For instance, the aromatic moiety may include a benzene moiety, a naphthalenyl moiety, an anthracenyl moiety, a pyridinyl moiety, a furanyl moiety, etc. Similarly, a “non-aromatic” structure is a structure in which aromaticity of the cyclic moiety is not present. For example, a non-aromatic cyclic structure may be a saturated cyclic structure, a cycloalkenyl moiety such as a cyclopentenyl moiety or a cyclohexenyl moiety, a cycloalkynyl moiety such as a cyclooctynyl moiety or a cyclodecynyl moiety, etc.

The cyclic moiety may include one or more substituents in certain cases, for example, attached to a ring within the cyclic moiety. The substituents may be any substituent, for example, as previously described in connection with alkyl moieties, for instance, a halogen such as fluorine, an alkoxy, an amine, a carbonyl, a hydroxide, or the like. In certain cases, a substituent on the cyclic moiety may be an alkyl moiety, as previously described (which itself may include one or more substituents, including other cyclic moieties).

In some cases, the humidity control material may include monomers or polymers in addition to those described above, for example, as in a co-polymer, a polymer blend, a multi-layered structure comprising the above-mentioned polymers in at least one layer, etc. Non-limiting examples of polymers that may be used within the humidity control material, in addition to the polymers described above, include polyfluoroorganic materials such as polytetrafluoroethylenes (e.g., such as those marketed under the name TEFLON® by DuPont of Wilmington, Del., for example, TEFLON® AF) or certain amorphous fluoropolymers; polystyrenes; polypropylenes (“PP”); silicones such as polydimethylsiloxanes; polysulfones; polycarbonates; acrylics such as polymethyl acrylate and polymethyl methacrylate; polyethylenes such as high-density polyethylenes (“HDPE”), low-density polyethylenes (“LDPE”), linear low-density polyethylenes (“LLDPE”), ultra low-density polyethylenes (“ULDPE”) etc.; PET; polyvinylchloride (“PVC”) materials; nylons; a thermoplastic elastomer; poly(1-trimethlsilyl-1-propyne) (“PTMSP”); and the like. Another example is poly(4-methylpentene-1) or poly(4-methyl-1-pentene) or poly(4-methyl-2-pentyne) (“PMP”): 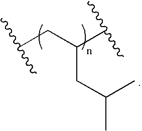
Examples of PMPs include those marketed under the name TPX™ by Mitsui Plastics (White Plains, N.Y.). As still another example, humidity control material may include poly(4-methylhexene-1), poly(4-methylheptene-1), poly(4-methyloctene-1), etc. In some cases, these materials may be copolymerized and/or in a polymer blend in association with the polymers as described above.

In some cases, the polymer (or mixture of polymers) used in the humidity control material may be sufficiently hydrophobic such that the polymer is able to retain water, i.e., water vapor is not able to readily transport through the polymer. For instance, the permeability of water through a hydrophobic polymer may be less than about 1000 (g micrometer/m² day), 900 (g micrometer/m² day), 800 (g micrometer/m² day), 600 (g micrometer/m² day) or less. The actual permeability of water through the polymer may also be a function of the relative humidity. In certain embodiments, the polymer(s) used in the humidity control material may have a molecular structure open enough to readily allow the transport of oxygen therethrough.

In another set of embodiments, the humidity control material may include a polymer that has bulky groups that prevent the polymer from readily forming a structure under ambient conditions that limits the transport of oxygen therethrough. A “bulky group” on a polymer, as used herein, is a moiety sufficiently large that the polymer forms a crystalline structure under ambient conditions that allows the transport of oxygen therethrough to greater than about 250 (cm³_{STP} mm/m² atm day). In some cases, the transport of oxygen may be greater than about 500 (cm³_{STP} mm/m² atm day), greater than about 1000 (cm³_{STP} mm/m² atm day), or greater than about 2000 (cm³_{STP} mm/m² atm day). The bulky group may be, for instance, part of the backbone of the polymer and/or part of a side chain. Non-limiting examples of bulky side groups include groups containing cyclopentyl moieties, isopropyl moieties, cyclohexyl moieties, phenyl moieties, isobutyl moieties, tert-butyl moieties, cycloheptyl moieties, trimethylsilyl or other trialkylsilyl moieties, etc. In some cases, the moiety may be halogenated, e.g. fluorinated, chlorinated, etc., as previously described. In one set of embodiments, the polymer may have a structure: 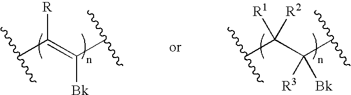
where each R independently comprises at least one atom, and Bk is a bulky group. In some cases, each R may independently be a hydrogen, a halogen, or an alkyl group. In some cases, Bk is halogenated. For example, Bk may comprise one or more fluorine atoms, chlorine atoms, bromines atoms, iodine atoms, etc. If more than one halogen atom is present, the halogen atoms may be the same or different.

Of course, it should be understood that the polymer may have several or all of the features described herein. For example, the polymer may be a polymer blend or a copolymer that has sufficient hydrophobicity such that the polymer is able to retain water, yet have a molecular structure open enough to allow sufficient oxygen permeability therethrough. In cases where the humidity control material includes multiple layers, some or all of the layers may also each include a mixture of materials in some embodiments. For example, one layer may include at least 50% by weight of one material with the balance comprising one or more other materials. In another embodiment, each layer consists essentially of a single material.

In one set of embodiments, the humidity control material may include and/or be fabricated in the form of a membrane or a thin film selected to control the passage of gases and/or water vapor therethrough. In one embodiment, the humidity controller is a membrane or a thin film having a desired permeability to one or more gases. The membrane or thin film may be positioned anywhere in the chip where it is able to affect one or more reaction sites in some fashion. For example, the membrane or thin film may be positioned such that it defines the surface of one or more reaction sites.

In one set of embodiments, the membrane or thin film has a thickness of greater than about 10 micrometers, in some cases greater than about 25 micrometers, in some cases greater than about 50 micrometers, in some cases greater than about 75 micrometers, in some cases greater than about 100 micrometers, in some cases greater than about 150 micrometers, in some cases greater than about 200 micrometers, or in some cases greater than about 250 micrometers, while still allowing sufficient oxygen transport therethrough, for instance, to enable cell culture of a plurality of aerobically respiring cells to occur, as further described herein. In some cases, a membrane or a thin film having a thickness of greater than about 50 micrometers may be particularly useful, for example, during manufacturing of a chip or other reaction system. In certain embodiments, the membrane may have a thickness of less than about 2 mm, less than about 1 mm, less than about 750 micrometers, less than about 500 micrometers, less than about 250 micrometers, or less than about 100 micrometers.

In some cases, it may be desired to incorporate the humidity control material into a structural components of the chip, reaction system, or other device, or to incorporate structural components of the chip, reaction system, or other device into the humidity control material. The humidity control material may also include a support layer, for example, where the humidity control material is intended to provide or supplement support, or will not itself be otherwise adequately supported. A support layer may comprise any material or materials that provides desired support. For example, the support layer may include one of the layers that may otherwise be included in the humidity control material for permeability, or the support layer may comprise a different material, such as glass (for example, PYREX® glass by Corning Glass of Corning, N.Y., or indium/tin-coated glass), latex, silicon, or the like. The support layer may be positioned anywhere within the humidity control material, for example, as an outer layer or an intermediate layer, and may be positioned to help protect one or more delicate layers. In some embodiments, the use of a support layer may allow a large portion, or nearly all of a reaction site, reactor, or chip to be constructed of the humidity control material. Preferably, the support layer does not significantly impact the permeability of the humidity control material, or the change in permeability may be accounted for in the design of the humidity control material.

Where the chip, reaction system, or other device of the present invention is intended for use with materials, such as reactants, that may damage, reduce the function, or otherwise react with or cause the humidity control material to deteriorate, the membrane may include a protection layer. The protection layer may be positioned, for example, as a surface layer, or interposed between a sensitive portion of the humidity control material and the material or environment that may adversely affect it. For example, the protection layer may be positioned on an inner surface of the humidity control material, particularly where the harmful material is within the chip, or on the outer surface of the humidity control material, particularly where the harmful material is outside the chip. The protection layer may also be positioned between other layers, so long as it is able to perform is protective function. Preferably, the protection layer does not significantly impact the permeability of the humidity control material. In some embodiments, any change in permeability may be accounted for in the design of the humidity control material.

As a non-limiting example, a chip 140 including a humidity controller according to one embodiment of the present invention is illustrated in FIG. 6A. This chip includes a reaction site 142, an inlet 144, an outlet 146, and an inner wall 148. Inner wall 148 is defined on one side by a humidity controller 150. Humidity controller 150, in this embodiment, includes a membrane having a first layer 152 and a second layer 154.

Another embodiment of a chip 140 including a humidity controller is illustrated in FIG. 6B. In this embodiment, the humidity controller 150 includes a multi-layer membrane that defines a wall of a reaction site 142, and also defines a wall of an inlet and of an outlet. In addition to first and second layers 152 and 154, which are provided primarily for purposes of providing a desired permeability, this membrane also includes a support layer 156 positioned between first and second layers 152, 154. Other arrangements for the permeability-controlling layer(s) and support layer(s) are possible. Also provided in chip 140 in this particular example is a cell adhesion layer 158 positioned on inner wall 148 of reaction site 142, encouraging cell growth there and not in inlet 144 and outlet 146. In other embodiments, the cell adhesion layer could extend over more, or all, of the surface of humidity controller 150. It should also be appreciated that the geometry of chip 140 as illustrated in FIGS. 6A and 6B is shown by way of illustration only and that many other arrangements and chip geometry may be useful in particular embodiments.

In one set of embodiments, the humidity control material is selected to have a certain permeability and/or a certain permeance. As used herein, the “permeability” of a material is given its ordinary meaning as used in the art, i.e., an intrinsic property that generally describes the ability of a gas to pass through the material. In contrast, as used herein, the “permeance” of a material is the actual rate of gas transport through a sample of a material, i.e., an extrinsic property. The permeance of a sample of material is affected by factors such as the area or thickness of the material, the pressure differential across the material, etc.

It should be appreciated that, while control of oxygen is used as an example herein, other gases such as nitrogen or carbon dioxide may be controlled instead, at permeabilities as noted above, or a combination of gases may be controlled. It should also be appreciated that while, in the example of cells further described below, the lower limit of oxygen transfer and the upper limit of water vapor transfer may typically be desired to be controlled, in other applications, for example, in a chemical synthesis operation, it may be desired to control other parameters, for example, the upper limit of oxygen transfer and lower limit of water vapor transfer, or the lower and upper limits of other gases such as nitrogen or carbon dioxide.

The presently disclosed humidity control materials may be used in a wide variety of reactions and interactions. One example of a reaction is cell culture, for example to maintain a cell culture, to increase the number of available cells or cell types, and/or to produce a desirable cellular product. In some cases, the humidity control material may allow sufficient oxygen to enter by diffusion therethrough to support cell growth, e.g., of aerobically respiring cells. In certain cases, the humidity control material may also be largely impermeable to microorganisms and other cells, for example, to prevent contamination. Preferably, the material has low toxicity.

In embodiments where chips, reaction systems, or other devices disclosed herein are used in connection with culturing cells, cell culture may take place over varying lengths of time, depending on the cells being cultured and other factors known to those of ordinary skill in the art. Thus, the design of the chip and the nature of the humidity control material may be adapted to the culture time. For example, the chip or humidity control material may be designed to allow it to maintain its desired functional characteristics for the time needed for the culture, and the humidity control material is preferably designed to be able to be reused many times. In various embodiments, cell culture may be performed over the course of 24 hours, 48 hours, 1 week, 2 weeks, 4 weeks, 6 weeks, 3 months, 1 year, continuously, or any other time required for a specific cell culture.

In some cases, the humidity control material is selected to have a permeability and/or a permeance to one or more gases that corresponds to a range acceptable for culturing certain cells, for example, aerobic cells. For example, the humidity control material may have a permeability and/or permeance to oxygen high enough, and/or a permeability and/or permeance to water vapor low enough, to allow cell culturing of specific cell types. Examples of such permeabilities include the above-described permeabilities. Those of skill in the art will be able to identify specific ranges of permeabilities of certain materials appropriate for successfully culturing particular cells and cell lines, as well as larger cellular groups, such as microbial cells, mammalian cells, tissues, tissue engineering constructs, etc.

Thus, in one embodiment, a method is provided that involves identifying an oxygen requirement and a humidity requirement of certain cells, selecting a material having an oxygen permeability high enough to meet the oxygen requirement of the cells and a water vapor permeability low enough to meet the humidity requirement of the cells, and culturing the cells in a chip, reaction system, or other device comprising a reaction site for such an embodiment. The reaction site has at least a portion thereof formed of the selected material.

In some embodiments, the humidity control material may not promote cell adhesion, but may include a cell adhesion layer (or a cell adhesion layer can be provided on the material) that may be any of a wide variety of hydrophilic, cytophilic, and/or biophilic materials. Examples of materials that may be suitable for a cell adhesion layer on a humidity control material include, but are not limited to, polyfluoroorganic materials, polyester, PDMS, polycarbonate, polystyrene, and aluminum oxide. As another example, the humidity control material may include a layer coated with a material that promotes cell adhesion, for example, using an RGD peptide sequence. In some embodiments, it may be desired to modify the surface of a cell adhesion layer, for example, by attachment, binding, soaking, or other treatments. Example molecules that promote cell adhesion include, but are not limited to, fibronectin, laminin, albumin, or collagen. Where the material includes a cell adhesion layer, the cell adhesion layer may be positioned as an inner layer or a surface layer of the membrane, may abut an interior of the chip, etc. Preferably, the cell adhesion layer does not significantly impact the permeability or permeance of the humidity control material. In some cases, any change in permeability or permeance may be accounted for in the design of the humidity control material.

Some of the materials used to form the humidity control material, and, in some cases, some of the layers thereof, may be selected based on the gas permeabilities of the materials, for example, as previously described. Those of ordinary skill in the art are aware of methods of determining the gas permeability of a material. As one particular method, a sample of a material having a known exposed area and thickness (e.g., a membrane) may be placed between two compartments, and a gas (or a liquid) may be placed in one compartment. The experimentally determined time it takes for the gas (or liquid) to diffuse across the material to the other compartment and be detected in a suitable fashion may then be related to the gas (or liquid) permeability of the material by standard, well-known mathematical transport relations. (See Examples 4-6 for a discussion and application of certain such mathematical transport relations as applied to the fabrication and performance of certain of the disclosed device embodiments).

In some embodiments, the area and thickness of the humidity control material, or a layer or portion thereof, may be used to select a desired degree of permeance and/or permeability. As one example, a more water vapor-permeable material may be made thicker, or its area may be reduced, in order to reduce the amount of water vapor that reaches and/or leaves the area or region where humidity control is desired. In some cases, the material may be designed such that it is between about 10 micrometers and 2 mm thick. Within this range, the relative thickness of layers within multiple layers or portions of the material may vary.

In one embodiment, a permeability goal may be achieved by combining two layers or portions of material. This can be achieved, for example, by including a first, more permeable layer, and a second, less permeable layer; multiple layers may also be used in other embodiments. By combining different materials and adjusting their relative thickness, a desired oxygen and water vapor permeability may be achieved. In one embodiment where the humidity control material comprises two layers or portions, they may be formed out of the same or different materials polymers. For example, the humidity control material may include a first layer including at least about 55% by weight of a first polymer or co-polymer and a second layer comprising no more than about 45% by weight of the first polymer or co-polymer. As another example, the humidity control material may include a first layer including at least about 60%, about 70%, or about 80% by weight of a first polymer or co-polymer and a second layer comprising no more than about 40%, about 30%, or about 20% by weight of the first polymer or copolymer. In some embodiments, the first polymer may comprise about 100% of the first layer and essentially none of the second layer. In some cases, at least a portion of the first layer may be co-polymerized with the second layer.

Where the humidity control material is constructed as a membrane including two or more layers, the two or more layers may be joined in any manner that provides sufficient strength to the membranes. In some cases, the two or more layers may be sufficiently self-supporting and it may not be necessary to join the layers, meaning a space could be left therebetween if desired. In other embodiments, additional layers may be used to support the membrane. In embodiments where it is desired to join the two or more layers to provide mutual support or otherwise, examples of acceptable means of joining the layers include laminating the layers together, at least partially intermixing the layers, and co-polymerizing the layers together. Where the layers are to be intermixed, the resin that will form each layer may be partially or totally intermixed before the membrane is formed. For example, liquid pre-polymers may be mixed and then a curing agent added, or two partially cured layers can be connected with a curing agent between them, curing the layers together.

In some embodiments, the presently disclosed humidity control materials allow light to pass through them. This may allow the materials to be used where light is important, for example, to facilitate a reaction such as a photocatalyzed reaction, to promote cell or plant growth, to cause a biochemical change to occur, or the like. The materials may also allow observation of a region, such as a reaction site, that is protected by a humidity control material, or is located behind a humidity-controlled region. In one embodiment, the humidity control material is translucent, and, in some cases, it is at least substantially transparent. One of skill in the art will recognize that there are varying degrees of translucence and transparence, and will be able to select desired properties based upon a particular application.

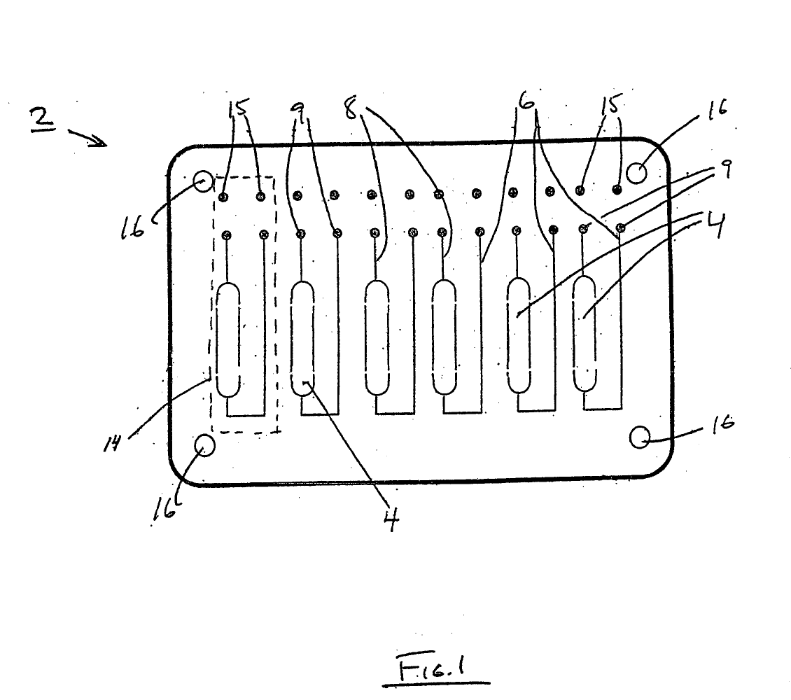

A variety of definitions will now be provided which will aid in understanding of the invention. Following, and interspersed with these definitions, is further disclosure, including descriptions of figures. Components shown in the figures that follow can generally be used in conjunction with layer 2 of FIG. 1. It is to be understood that in FIG. 1, and in all of the other figures, the arrangement of reaction sites, number of reaction sites, arrangement of channels addressing reaction sites, ports, and the like are merely given as examples that fall within the overall invention.

A “chemical, biological, or biochemical reactor chip,” (also referred to, equivalently, simply as a “chip”) as used herein, is an integral article that includes one or more reactors. “Integral article” means a single piece of material, or assembly of components integrally connected with each other. As used herein, the term “integrally connected,” when referring to two or more objects, means objects that do not become separated from each other during the course of normal use, e.g., cannot be separated manually; separation requires at least the use of tools, and/or by causing damage to at least one of the components, for example, by breaking, peeling, etc. (separating components fastened together via adhesives, tools, etc.).

A chip can be connected to or inserted into a larger framework defining an overall reaction system, for example, a high-throughput system. The system can be defined primarily by other chips, chassis, cartridges, cassettes, and/or by a larger machine or set of conduits or channels, sources of reactants, cell types, and/or nutrients, inlets, outlets, sensors, actuators, and/or controllers. Typically, the chip can be a generally flat or planar article (i.e., having one dimension that is relatively small compared to the other dimensions); however, in some cases, the chip can be a non-planar article, for example, the chip may have a cubical shape, a curved surface, a solid or block shape, etc.

As used herein, a “membrane” is a thin sheet of material, typically having a shape such that one of the dimensions is substantially smaller than the other dimensions, that is permeable to at least one substance in an environment to which it is or can be exposed. In some cases, the membrane may be generally flexible or non-rigid. As an example, a membrane may be a rectangular or circular material with a length and width on the order of millimeters, centimeters, or more, and a thickness of less than a millimeter, and in some cases, less than 100 microns, less than 10 microns, or less than 1 micron or less. The membrane may define a portion of a reaction site and/or a reactor, or the membrane may be used to divide a reaction site into two or more portions, which may have volumes or dimensions which are substantially the same or different. Non-limiting examples of substances to which the membrane may be permeable to include water, O₂, CO₂, or the like. As an example, a membrane may have a permeability to water of less than about 1000 (g micrometer/m² day), 900 (g micrometer/m² day), 800 (g micrometer/m² day), 600 (g micrometer/m² day) or less; the actual permeability of water through the membrane may also be a function of the relative humidity in some cases.

Some membranes may be semipermeable membranes, which those of ordinary skill in the art will recognize to be membranes permeable with respect to at least one species, but not readily permeable with respect to at least one other species. For example, a semipermeable membrane may allow oxygen to permeate across it, but not allow water vapor to do so, or may allow water vapor to permeate across it, but at a rate that is at least an order of magnitude less than that for oxygen. Or a semipermeable membrane may be selected to allow water to permeate across it, but not certain ions. For example, the membrane may be permeable to cations and substantially impermeable to anions, or permeable to anions and substantially impermeable to cations (e.g., cation exchange membranes and anion exchange membranes). As another example, the membrane may be substantially impermeable to molecules having a molecular weight greater than about 1 kilodalton, 10 kilodaltons, or 100 kilodaltons or more. In one embodiment, the membrane may be impermeable to cells, but be chosen to be permeable to varied selected substances; for example, the membrane may be permeable to nutrients, proteins and other molecules produced by the cells, waste products, or the like. In other cases, the membrane may be gas impermeable. Some membranes may be transparent to particular light (e.g. infrared, UV, or visible light; light of a wavelength with which a device utilizing the membrane interacts; visible light if not otherwise indicted). Where a membrane is substantially transparent, it absorbs no more than 50% of light, or in other embodiments no more than 25% or 10% of light, as described more fully herein. In some cases, a membrane may be both semipermeable and substantially transparent. The membrane, in one embodiment, may be used to divide a reaction site constructed and arranged to support cell culture from a second portion, for example, a reservoir. For example, a reaction site may be divided into three portions, four portions, or five portions. For instance, a reaction site may be divided into a first cell culture portion and a second cell culture portion flanking a first reservoir portion and two additional reservoir portions, one of which is separated by a membrane from the first cell culture portion and the other of which is separated by a membrane from the second cell culture portion. Of course, those of ordinary skill in the art will be able to design other arrangements, having varying numbers of cell culture portions, reservoir portions, and the like, as described herein.

As used herein, a “substantially transparent” material (for example, a membrane) is a material that is able to transmit electromagnetic radiation in some cases such that a majority of the radiation incident on the material passes through the material unaltered, and in some embodiments, at least about 75%, in other embodiments at least about 80%, in still other embodiments at least about 90%, in still other embodiments at least about 95%, in still other embodiments at least about 97%, and in still other embodiments at least about 99% of the incident radiation is able to pass through the material unaltered. In certain cases, the material is at least partially transparent to electromagnetic radiation within the above-mentioned wavelength range to the extent necessary to promote and/or monitor a physical, chemical, biochemical, and/or biological reaction occurring within a reaction site, for example as previously described. In other embodiments, the material may be transparent to electromagnetic radiation within the above-mentioned wavelength range to the extent necessary to monitor, observe, stimulate and/or control a cell within the reaction site. In some cases, the material is substantially transparent to incident electromagnetic radiation ranging between the infrared and ultraviolet ranges (including visible light) and, in particular, between wavelengths of about 400 nm-410 nm and about 1,000 nm. In some cases, the material may be transparent to electromagnetic radiation between wavelengths of about 400 nm-410 nm and about 800 nm, and in some embodiments, the material may be substantially transparent to radiation between wavelengths of about 450 nm and 700 nm.

As used herein, a “reactor” is the combination of components including a reaction site, any chambers (including reaction chambers and ancillary chambers), channels, ports, inlets and/or outlets (i.e., leading to or from a reaction site), sensors, actuators, processors, controllers, membranes, and the like, which, together, operate to contain, promote and/or monitor a biological, chemical, and/or biochemical reaction, interaction, operation, or experiment at a reaction site, and which can be part of a chip. For example, a chip may include at least 5, at least 10, at least 20, at least 50, at least 100, at least 500, or at least 1,000 or more reactors. Examples of reactors include chemical or biological reactors and cell culturing devices, as well as the reactors described in International Patent Application No. PCT/US01/07679, filed Mar. 9, 2001, entitled “Microreactor,” by Jury, et al., published as WO 01/68257 on Sep. 20, 2001, incorporated herein by reference. Reactors can include one or more reaction sites or compartments. The reactor may be used for any chemical, biochemical, and/or biological purpose, for example, cell growth, pharmaceutical production, chemical synthesis, hazardous chemical production, drug screening, materials screening, drug development, chemical remediation of warfare reagents, or the like. For example, the reactor may be used to facilitate very small scale culture of cells or tissues. In one set of embodiments, a reactor of the invention comprises a matrix or substrate of a few millimeters to centimeters in size, containing channels with dimensions on the order of, e.g., tens or hundreds of micrometers. Reagents of interest may be allowed to flow through these channels, for example to a reaction site, or between different reaction sites, and the reagents may be mixed or reacted in some fashion. The products of such reactions can be recovered, separated, and treated within the reactor or chip in certain cases.

As used herein, a “reaction site” is defined as a site within a reactor that is constructed and arranged to produce a physical, chemical, biochemical, and/or biological reaction during use of the reactor. More than one reaction site may be present within a reactor or a chip in some cases, for example, at least one reaction site, at least two reaction sites, at least three reaction sites, at least four reaction sites, at least 5 reaction sites, at least 7 reaction sites, at least 10 reaction sites, at least 15 reaction sites, at least reaction sites, at least 30 reaction sites, at least 40 reaction sites, at least 50 reaction sites, at least 100 reaction sites, at least 500 reaction sites, or at least 1,000 reaction sites or more may be present within a reactor or a chip. The reaction site may be defined as a region where a reaction is allowed to occur; for example, a reactor may be constructed and arranged to cause a reaction within a channel, one or more compartments, at the intersection of two or more channels, etc. The reaction may be, for example, a mixing or a separation process, a reaction between two or more chemicals, a light-activated or a light-inhibited reaction, a biological process, and the like. In some embodiments, the reaction may involve an interaction with light that does not lead to a chemical change, for example, a photon of light may be absorbed by a substance associated with the reaction site and converted into heat energy or re-emitted as fluorescence. In certain embodiments, the reaction site may also include one or more cells and/or tissues. Thus, in some cases, the reaction site may be defined as a region surrounding a location where cells are to be placed within the reactor, for example, a cytophilic region within the reactor.

In some cases, the reaction site containing cells may include a region containing a gas (e.g., a “gas head space”), for example, if the reaction site is not completely filled with a liquid. The gas head space, in some cases, may be partially separated from the reaction site, through use of a gas-permeable or semi-permeable membrane. In some cases, the gas head space may include various sensors for monitoring temperature, and/or other reaction conditions.

Many embodiments and arrangements of the disclosed devices are described with reference to a chip, or to a reactor, and those of ordinary skill in the art will recognize that the presently disclosed subject matter can apply to either or both. For example, a channel arrangement may be described in the context of one, but it will be recognized that the arrangement can apply in the context of the other (or, typically, both: a reactor which is part of a chip). It is to be understood that all descriptions herein that are given in the context of a reactor or chip apply to the other, unless inconsistent with the description of the arrangement in the context of the definitions of “chip” and “reactor” herein.

The term “determining,” as used herein, generally refers to the measurement and/or analysis of a substance (e.g., within a reaction site), for example, quantitatively or qualitatively, or the detection of the presence or absence of the substance. “Determining” may also refer to the measurement and/or analysis of an interaction between two or more substances, for example, quantitatively or qualitatively, or by detecting the presence or absence of the interaction. Examples of techniques suitable for “determining” include, but are not limited to, gravimetric analysis, calorimetry, pressure or temperature measurement, spectroscopy such as infrared, absorption, fluorescence, UV/visible, FTIR (“Fourier Transform Infrared Spectroscopy”), or Raman spectroscopy; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; or turbidity measurements, including nephelometry.

Exemplary features of certain disclosed devices are now described in more detail. Referring to FIG. 1, one portion of a chip according to one embodiment is illustrated schematically. The portion illustrated is a layer 2 which includes within it a series of void spaces which, when layer 2 is positioned between two layers (a top and bottom layer relative to the plane of FIG. 1, not shown) define a series of enclosed channels and reaction sites. The overall arrangement into which layer 2 can be assembled to form a chip will be understood more clearly from the description below with respect to other figures.

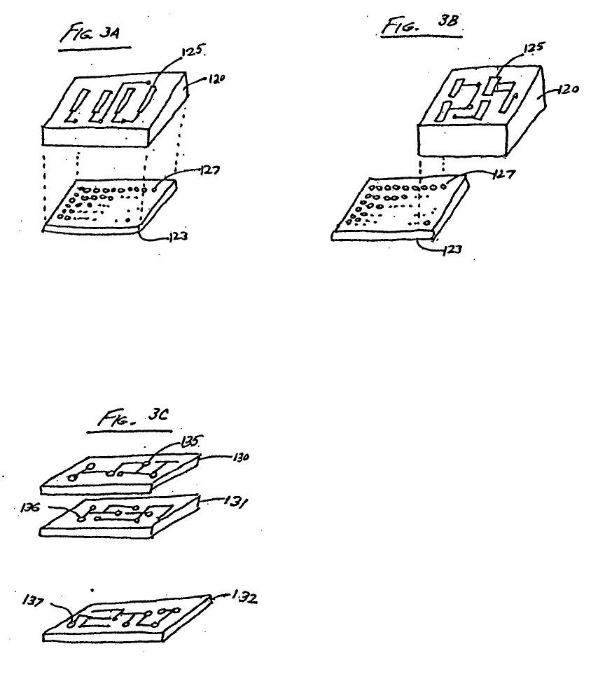

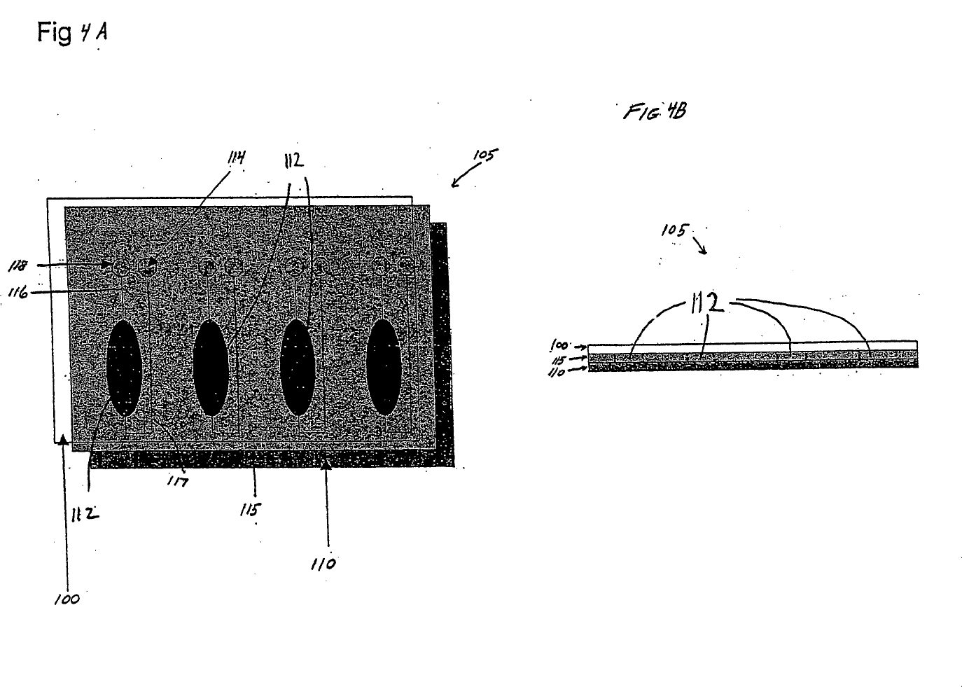

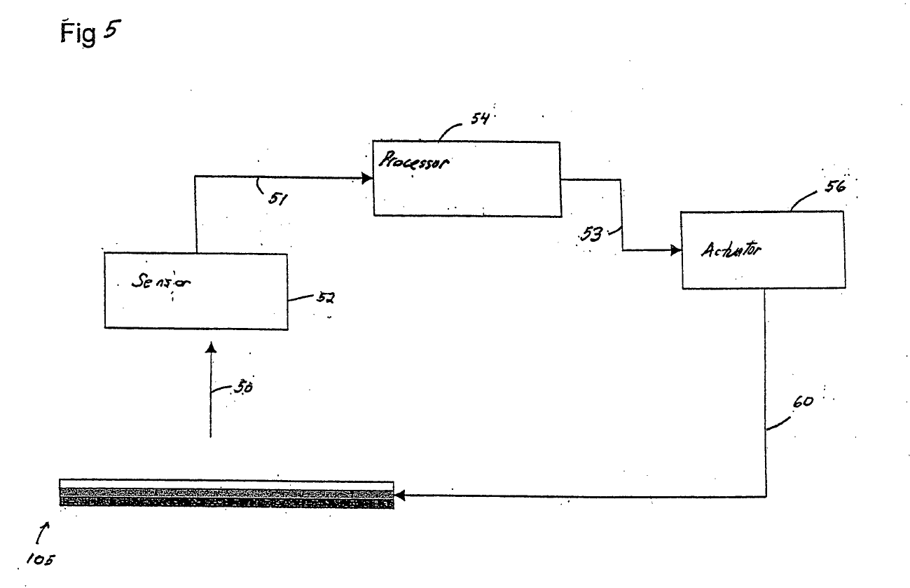

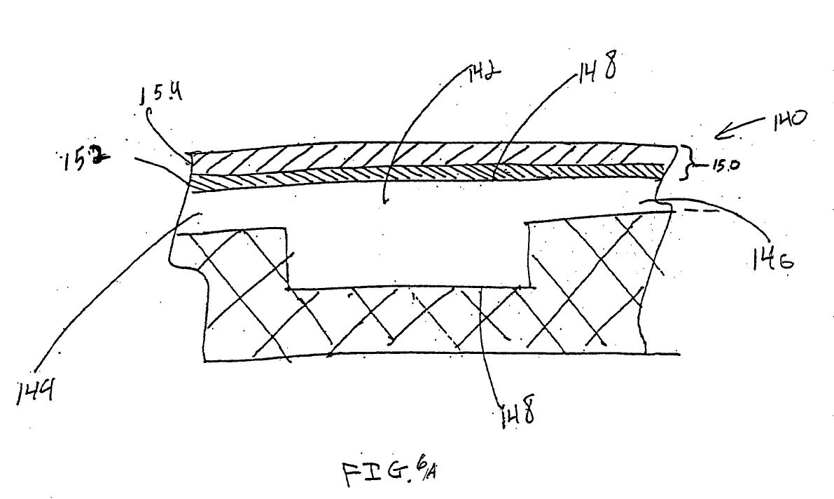

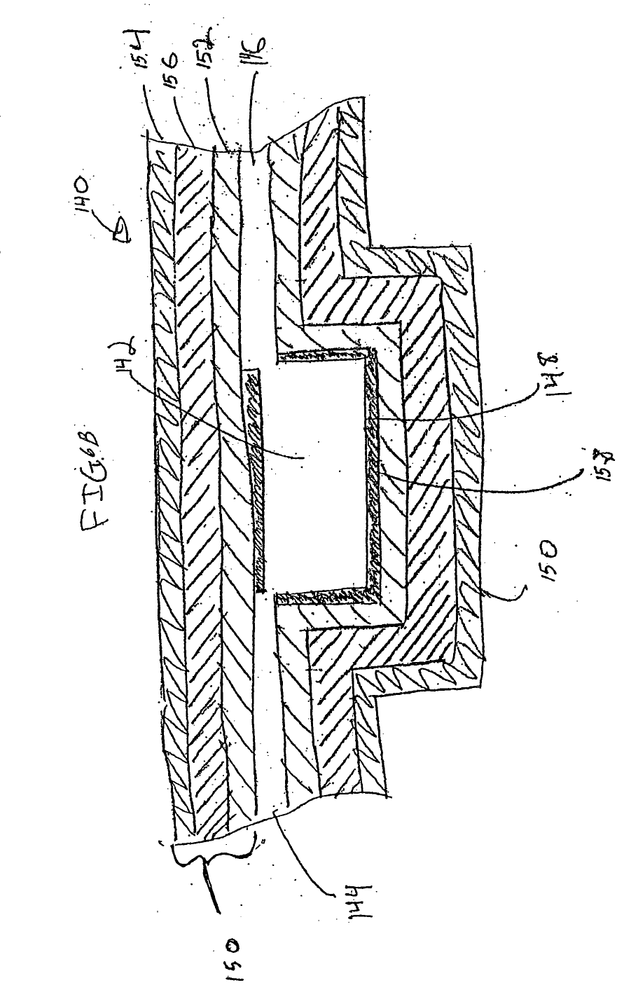

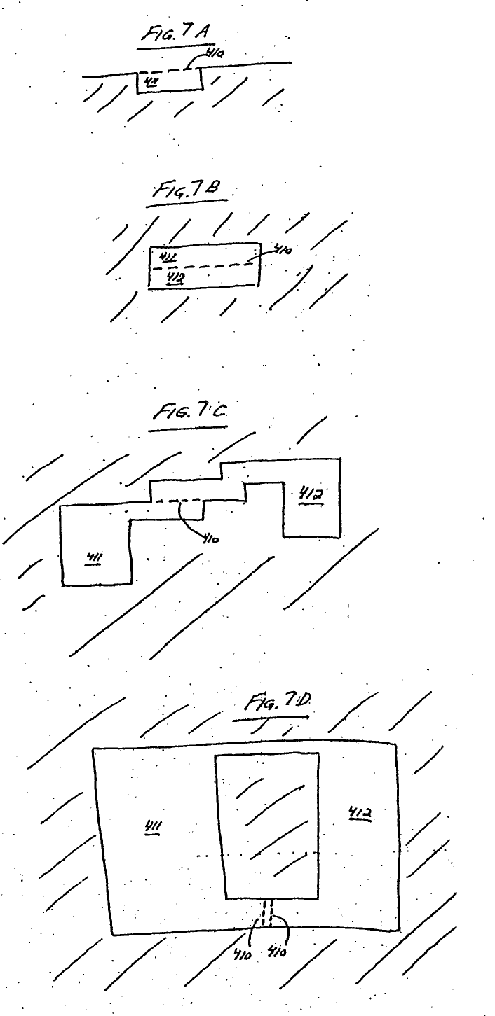

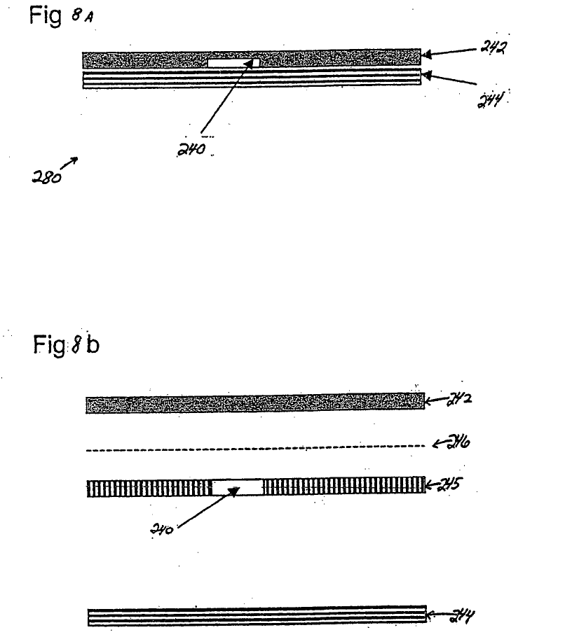

FIG. 1 represents an embodiment including six reaction sites 4 (analogous to, for example, reaction site 125 of FIG. 3A, or reaction site 112 of FIG. 4A, described below). Reaction sites 4, in certain embodiments, can define a series of generally aligned, elongated, rounded rectangular voids within a relatively thin, generally planar piece of material defining layer 2. Reaction sites 4 can be addressed by a series of channels including channels 6 for delivering species to reaction sites 4 and channels 8 for removal of species from the reaction sites. Of course, any combination of channels can be used to deliver and/or remove species from the reaction sites. For example, channels 8 can be used to deliver species to the reaction sites while channels 6 can be used to remove species, etc. Although shown as lines in FIG. 1, channels 6 and 8 are to be understood to define voids within layer 2 which, when covered above and/or below by other layers, may become enclosed channels. Each of channels 6 and 8, in the embodiment illustrated in FIG. 1, is addressed by a port 9. Where port 9 is connected to an inlet channel it can define an inlet port, and where fluidly connected to an outlet channel it can define an outlet port. In the embodiment illustrated, port 9 is a void that is larger in width than the width of channels 6 or 8. Those of ordinary skill in the art will recognize a variety of techniques for accessing ports 9 and utilizing them to introduce species into channels, and/or remove species from channels addressed by those ports. As one example, port 9 can be a “self-sealing” port, addressable by a needle (as described more fully below) when at least one side of port 9 is covered by a layer (not shown) of material which, when a needle is inserted through the material and withdrawn, forms a seal generally impermeable to species such as fluids introduced into or removed from the chip via the port.

Also shown in FIG. 1 are a series of ports 15, not shown to be fluidly connected or connectable to any inlet channels, outlet channels, or reaction sites of the chip. Ports 15 can be defined by voids in layer 2, and can be used to facilitate fluidic connection between and among various layers of a chip and/or an environment external to the chip. As an example, where layer 2 forms part of a multi-layer chip including multiple reaction sites in different layers, another layer may be provided on one side of layer 2 (optionally separated by an intermediate layer or layers) including one set of reaction sites or conduits, and another layer may be provided on the opposite side of layer 2, similarly separated by intermediate layers if desirable, and ports 15 may define passages or routes for fluidic connection between reaction sites and/or conduits of chip layers on opposite sides of layer 2. Ports 15 also may connect to channels communicating with a compartment aligned with a compartment defining reaction site 4, separated from the reaction site by a membrane, e.g. semipermeable membrane. In this way, fluid can be independently flowed into, out of, and/or through a space on one side of a membrane, and also independently through a space on the other side of the membrane, one or both defining a compartment and/or reaction site.

In FIG. 1, each reaction site 4, along with the associated fluidic connections (e.g., channels 6 and 8, ports 9 and ports 15), together define a reactor 14, as indicated by dotted lines. In FIG. 1, layer 2 contains six such reactors, each reactor having substantially the same configuration. In other embodiments, a reactor may include more than one reaction site, channels, ports, etc. Additionally, a chip layer may have reactors that do not have substantially the same configuration.

Additionally shown in FIG. 1 is a series of devices 16 which can be used to secure layer 2 to other layers of a chip and/or to assure alignment of layer 2 with other layers and/or other systems to which the chip is desirably coupled. Devices 16 can define screws, posts, indentations (i.e., that match corresponding protrusions of other layers or devices), or the like. Those of ordinary skill in the art are aware of a variety of suitable techniques for securing layers to other layers and/or chips disclosed herein to other components or systems using devices such as these.

In some embodiments, the reaction site(s) of a device, such as a chip or reactor, may be defined by geometrical considerations. For example, the reaction site may be defined as a compartment in a reactor, a channel, an intersection of two or more channels, or other location defined in some fashion (e.g., formed or etched within a substrate that can define a reactor and/or chip). Other methods of defining a reaction site are also possible. In some embodiments, the reaction site may be artificially created, for example, by the intersection or union of two or more fluids (e.g., within one or several channels), or by constraining a fluid on a surface, for example, using bumps or ridges on the surface to constrain fluid flow. In other embodiments, the reaction site may be defined through electrical, magnetic, and/or optical systems. For example, a reaction site may be defined as the intersection between a beam of light and a fluid channel.

The volume of the reaction site can be very small in certain embodiments. Specifically, the reaction site may have a volume of less than one liter, less than about 100 ml, les than about 10 ml, less than about 5 ml, less than about 3 ml, less than about 2 ml, less than about 1 ml, less than about 500 microliters, less than about 300 microliters, less than about 200 microliters, less than about 100 microliters, less than about 50 microliters, less than about 30 microliters, less than about 20 microliters or less than about 10 microliters in various embodiments. The reaction site may also have a volume of less than about 5 microliters, or less than about 1 microliter in certain cases. The reaction site may have any convenient size and/or shape. In another set of embodiments, the reaction site may have a dimension that is 500 microns deep or less, 200 microns deep or less, or 100 microns deep or less.

In some cases, cells can be present at the reaction site. Sensor(s) associated with the chip or reactor, in certain cases, may be able to determine the number of cells, the density of cells, the status or health of the cell, the cell type, the physiology of the cells, etc. In certain cases, the reactor can also maintain or control one or more environmental factors associated with the reaction site, for example, in such a way as to support a chemical reaction or a living cell. In one set of embodiments, a sensor may be connected to an actuator and/or a microprocessor able to produce an appropriate change in an environmental factor within the reaction site. The actuator may be connected to an external pump, the actuator may cause the release of a substance from a reservoir, or the actuator may produce sonic or electromagnetic energy to heat the reaction site, or selectively kill a type of cell susceptible to that energy. The reactor can include one or more than one reaction site, and one or more than one sensor, actuator, processor, and/or control system associated with the reaction site(s). It is to be understood that any reaction site or a sensor technique disclosed herein can be provided in combination with any combination of other reaction sites and sensors.

As used herein, a “channel” is a conduit associated with a reactor and/or a chip (within, leading to, or leading from a reaction site) that is able to transport one or more fluids specifically from one location to another, for example, from an inlet of the reactor or chip to a reaction site, e.g., as further described below. Materials (e.g., fluids, cells, particles, etc.) may flow through the channels, continuously, randomly, intermittently, etc. The channel may be a closed channel, or a channel that is open, for example, open to the external environment surrounding the reactor or chip containing the reactor. The channel can include characteristics that facilitate control over fluid transport, e.g., structural characteristics (e.g., an elongated indentation), physical/chemical characteristics (e.g., hydrophobicity vs. hydrophilicity) and/or other characteristics that can exert a force (e.g., a containing force) on a fluid when within the channel. The fluid within the channel may partially or completely fill the channel. In some cases the fluid may be held or confined within the channel or a portion of the channel in some fashion, for example, using surface tension (i.e., such that the fluid is held within the channel within a meniscus, such as a concave or convex meniscus). The channel may have any suitable cross-sectional shape that allows for fluid transport, for example, a square channel, a circular channel, a rounded channel, a rectangular channel (e.g., having any aspect ratio), a triangular channel, an irregular channel, etc. The channel may be of any size within the reactor or chip. For example, the channel may have a largest dimension perpendicular to a direction of fluid flow within the channel of less than about 1000 micrometers in some cases, less than about 500 micrometers in other cases, less than about 400 micrometers in other cases, less than about 300 micrometers in other cases, less than about 200 micrometers in still other cases, less than about 100 micrometers in still other cases, or less than about 50 or 25 micrometers in still other cases. In some embodiments, the dimensions of the channel may be chosen such that fluid is able to freely flow through the channel, for example, if the fluid contains cells. The dimensions of the channel may also be chosen in certain cases, for example, to allow a certain volumetric or linear flowrate of fluid within the channel. In one embodiment, the depth of other largest dimension perpendicular to a direction of fluid flow may be similar to that of a reaction site to which the channel is in fluid communication with. Of course, the number of channels, the shape or geometry of the channels, and the placement of channels within the chip can be determined by those of ordinary skill in the art.

Devices, such as chips, disclosed herein may also include a plurality of inlets and/or outlets that can receive and/or output any of a variety of reactants, products, and/or fluids, for example, directed towards one or more reactors and/or reaction sites. In some cases, the inlets and/or outlets may allow the aseptic transfer of compounds. At least a portion of the plurality of inlets and/or outlets may be in fluid communication with one or more reaction sites within the device. In some cases, the inlets and/or outlets may also contain one or more sensors and/or actuators, as further described below. Essentially, the device or chip may have any number of inlets and/or outlets from one to tens of hundreds that can be in fluid communication with one or more reactors and/or reaction sites. Less than 5 or 10 inlets and/or outlets may be provided to the reactor and/or reaction site(s) for certain reactions, such as biological or biochemical reactions. In some cases each reactor may have around 25 inlets and/or outlets, in other cases around 50 inlets and/or outlets, in still other cases around 75 inlets and/or outlets, or around 100 or more inlets and/or outlets in still other cases.

As one example, the inlets and/or outlets of the device, directed to one or more reactors and/or reaction sites may include inlets and/or outlets for a fluid such as a gas or a liquid, for example, for a waste stream, a reactant stream, a product stream, an inert stream, etc. In some cases, the device may be constructed and arranged such that fluids entering or leaving reactors and/or reaction sites do not substantially disturb reactions that may be occurring therein. For example, fluids may enter and/or leave a reaction site without affecting the rate of reaction in a chemical, biochemical, and/or biological reaction occurring within the reaction site, or without disturbing and/or disrupting cells that may be present within the reaction site. Examples of inlet and/or outlet gases may include, but are not limited to, CO₂, CO, oxygen, hydrogen, NO, NO₂, water vapor, nitrogen, ammonia, acetic acid, etc. As another example, an inlet and/or outlet fluid may include liquids and/or other substances contained therein, for example, water, saline, cells, cell culture medium, blood or other bodily fluids, antibodies, pH buffers, solvents, hormones, carbohydrates, nutrients, growth factors, therapeutic agents (or suspected therapeutic agents), antifoaming agents (e.g., to prevent production of foam and bubbles), proteins, antibodies, and the like. The inlet and/or outlet fluid may also include a metabolite in some cases. A “metabolite,” as used herein, is any molecule that can be metabolized by a cell. For example, a metabolite may be or include an energy source such as a carbohydrate or a sugar, for example, glucose, fructose, galactose, starch, corn syrup, and the like. Other example metabolites include hormones, enzymes, proteins, signaling peptides, amino acids, etc.

The inlets and/or outlets may be formed within the device by any suitable technique known to those of ordinary skill in the art, for example, by holes or apertures that are punched, drilled, molded, milled, etc. within the device or within a portion of the device, such as a substrate layer of a chip. In some cases, the inlets and/or outlets may be lined, for example, with an elastomeric material. In certain embodiments, the inlets and/or outlets may be constructed using self-sealing materials that may be re-usable in some cases. For example, an inlet and/or outlet may be constructed out of a material that allows the inlet and/or outlet to be liquid-tight (i.e., the inlet and/or outlet will not allow a liquid to pass therethrough without the application of an external driving force, but may admit the insertion of a needle or other mechanical device able to penetrate the material under certain conditions). In some cases, upon removal of the needle or other mechanical device, the material may be able to regain its liquid-tight properties (i.e., a “self-sealing” material). Non-limiting examples of self-sealing materials suitable for use with the invention include, for example, polymers such as polydimethylsiloxane (“PDMS”), natural rubber, HDPE, or silicone materials such as Formulations RTV 108, RTV 615, or RTV 118 (General Electric, New York, N.Y.).

In some embodiments, the disclosed device may include very small elements, for example, sub-millimeter or microfluidic elements. For example, in some embodiments, the devices may include at least one reaction site having a cross sectional dimension of no greater than, for example, 100 mm, 80 mm, 50 mm, or 10 mm. In some embodiments, the reaction site may have a maximum cross section no greater than, for example, 100 mm, 80 mm, 50 mm, or 10 mm. As used herein, the “cross section” refers to a distance measured between two opposed boundaries of the reaction site, and the “maximum cross section” refers to the largest distance between two opposed boundaries that may be measured. In other embodiments, a cross section or a maximum cross section of a reaction site may be less than 5 mm, less than 2 mm, less than 1 mm, less than 500 micrometers, less than 300 micrometers, less than 100 micrometers, less than 10 micrometers, or less than 1 micrometer or smaller. As used herein, a “microfluidic chip” is a chip comprising at least one fluidic element having a sub-millimeter cross section, i.e., having a cross section that is less than 1 mm. As one particular non-limiting example, a reaction site may have a generally rectangular shape, with a length of 80 mm, a width of 10 mm, and a depth of 5 mm.

While one reaction site may be able to hold and/or react a small volume of fluid as described herein, the technology associated with the invention also allows for scalability and parallelization. With regard to throughput, an array of many reactors and/or reaction sites within a chip or other device, or within a plurality of chips or other devices, can be built in parallel to generate larger capacities. For example, a plurality of chips (e.g. at least about 10 chips, at least about 30 chips, at least about 50 chips, at least about 75 chips, at least about 100 chips, at least about 200 chips, at least about 300 chips, at least about 500 chips, at least about 750 chips, or at least about 1,000 chips or more) may be operated in parallel, for example, through the use of robotics, for example which can monitor or control the chips automatically. Additionally, an advantage may be obtained by maintaining production capacity at the small scale of reactions typically performed in the laboratory, with scale-up via parallelization. It is a feature of certain embodiments that many reaction sites may be arranged in parallel within a reactor of a chip and/or within a plurality of chips. Specifically, in certain embodiments, at least five reaction sites can be constructed to operate in parallel, or in other cases at least about 7, about 10, about 30, about 50, about 100, about 200, about 500, about 1,000, about 5,000, about 10,000, about 50,000, or even about 100,000 or more reaction sites can be constructed to operate in parallel, for example, in a high-throughput system. In some cases, the number of reaction sites may be selected so as to produce a certain quantity of a species or product, or so as to be able to process a certain amount of reactant. In certain cases the parallelization of the chips and/or reactors may allow many compounds to be screened simultaneously, or many different growth conditions and/or cell lines to be tested and/or screened simultaneously. Of course, the exact locations and arrangement of the reaction site(s) within the reactor or chip will be a function of the specific application.

Additionally, certain embodiments described herein may be configured to be used in conjunction with a collection chamber connectable ultimately to an outlet of one or more reactors and/or reaction sites of a chip. The collection chamber may have a volume of greater than 10 milliliters or 100 milliliters in some cases. The collection chamber, in other cases, may have a volume of greater than 100 liters or 500 liters, or greater than 1 liter, 2 liters, 5 liters, or 10 liters. Large volumes may be appropriate where the reactors and/or reaction sites are arranged in parallel within one or more chips, e.g., a plurality of reactors and/or reaction sites may be able to deliver a product to a collection chamber.

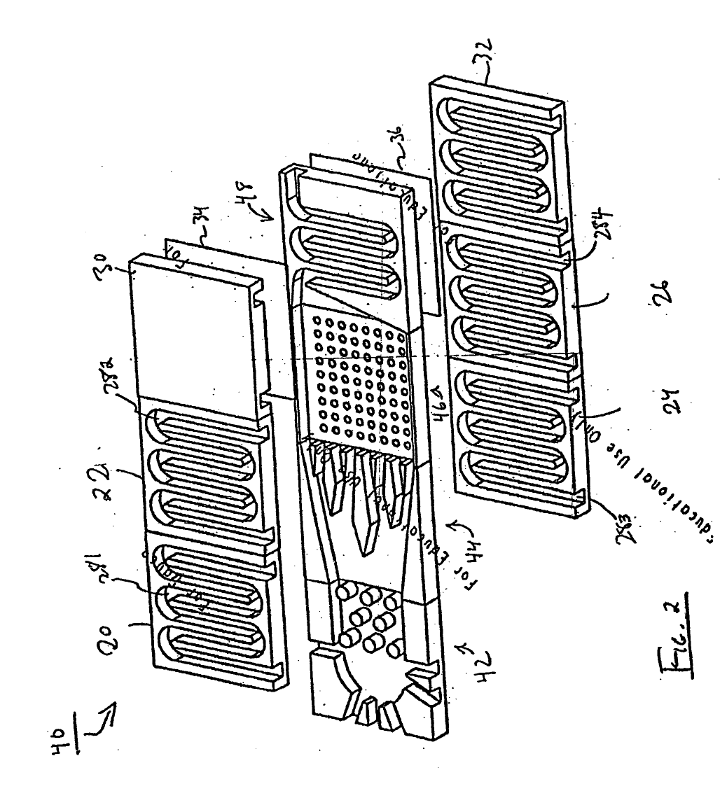

In some embodiments, the reaction site(s) and/or the channels in fluidic communication with the reaction site(s) are free of active mixing elements. In these embodiments, the reactor of the chip can be constructed in such a way as to cause turbulence in the fluids provided through the inlets and/or outlets, thereby mixing and/or delivering a mixture of the fluids, preferably without active mixing, where mixing is desired. Specifically, the reactor and/or reaction site(s) may include a plurality of obstructions in the flow path of the fluid that causes fluid flowing through the flow path to mix, for example, as shown in mixing unit 42 in FIG. 2. These obstructions can be of essentially any geometrical arrangement for example, a series of pillars. As used herein, “active mixing elements” is meant to define mixing elements such as blades, stirrers, or the like, which are movable relative to the reaction site(s) and/or channels themselves, that is, movable relative to portion(s) of the reactor defining a reaction site a or a channel.

Chips of certain embodiments may be constructed and arranged such that they are able to be stacked in a predetermined, pre-aligned relationship with other, similar chips, such that the chips are all oriented in a predetermined way (e.g., all oriented in the same way) when stacked together. When a chip is designed to be stacked with other, similar chips, the chip often can be constructed and arranged such that at least a portion of the chip, such as a reaction site, is in fluidic communication with one or more of the other chips and/or reaction sites within other chips. This arrangement may find use in parallelization of chips, as discussed herein.

In one set of embodiments, a presently disclosed chip is constructed and arranged such that the chip is able to be stably connected to a microplate, for example, as defined in the 2002 SPS/ANSI proposed standard (e.g., a microplate having dimensions of roughly 127.76±0.50 mm by 85.48±0.50 mm). As used herein, “stably connected” refers to systems in which two components are connected such that a specific motion or force is necessary to disconnect the two components from each other, i.e., the two components cannot be dislodged by random vibration or displacement (e.g., accidentally lightly bumping a component). The components can be stably connected by way of pegs, screws, snap-fit components, matching sets of indentations and protrusions, or the like. A “microplate” is also sometimes referred to as a “microtiter” plate, a “microwell” plate, or other similar terms known to the art. The microplate may include any number of wells. For example, as is typically used commercially, the microplate may be a six-well microplate, a 24-well microplate, a 96-well microplate, a 384-well microplate, or a 1,536-well microplate. The wells may be of any suitable shape, for example, cylindrical or rectangular. The microplate may also have other numbers of wells and/or other well geometries or configurations, for instance, in certain specialized applications.

FIGS. 3A-3C illustrate one set of embodiments in which one or more reaction sites may be positioned in association with a chip such that, when the chip is stably connected to other chips and/or microplates, one or more reaction sites of the chip are positioned or aligned to be in chemical, biological, or biochemical communication with, or chemically, biologically, or biochemically connectable with one or more reaction sites of the other chip(s) and/or one or more wells of the microplate(s). “Alignment,” in this context, can mean complete alignment, such that the entire area of the side of a reaction site adjacent another reaction site or well completely overlaps the other reaction site or well, and vice versa, or partial alignment, where at least a portion of the reaction site can overlap at least a portion of an adjacent reaction site or well. “Chemically, biologically, or biochemically connectable” means that the reaction site is in fluid communication with another reaction site or well (i.e., fluid is free to flow from one to the other); or is fluidly connectable to the other site or well (e.g., the two are separated from each other by a wall or other component that can be punctured or ruptured, or by a valve in a conduit connecting the two that can be opened); or the reaction site and other site or well are arranged such that at least some chemical, biological, or biochemical species can migrate from one to the other, e.g., across a semipermeable membrane. As examples, a chip may have six reaction sites that are constructed and arranged to be aligned with the six wells of a 6-well microplate when the chip is stably connected with the microplate (e.g., positioned on top of the microplate), a chip having 96 reaction sites may be constructed and arranged such that the 96 reaction sites are constructed and arranged to be aligned with the 96 wells of a 96-well microplate when the chip is stably connected with the microplate, etc. Of course, in some cases, the chip may be constructed and arranged such that a single reaction site of the chip is aligned with more than one microplate well and/or more than one other reaction site on the microplate well, and/or such that more than one microplate well and/or more than one other reaction site on the microplate well is aligned with a single reaction site of the chip.

Disclosed chips also may be constructed and arranged such that at least one reaction site and/or reactor of the chip is in fluid communication with, and/or chemically, biologically, or biochemically connectable to an apparatus constructed and arranged to address at least one well of a microplate, for example, an apparatus that can add species to and/or remove species from wells of microplates, and/or can test species within wells of a microplate. In this arrangement, the apparatus may add and/or remove species to/from a reaction site of a chip, and/or test species at reaction sites. In this embodiment, the reaction sites typically are arranged in alignment with wells of the microplate.

With reference to FIGS. 3A and 3B, examples are shown in which chip 120 may be stably connected to commercially-available microplate 123. In FIG. 3A, chip 120 may be positioned such that at least some of reaction sites 125 of chip 120 are aligned with, and/or connectable with at least some of wells 127 of microplate 123 when chip 120 is stably connected to microplate 123. Similarly, in FIG. 3B, chip 120 may be constructed and arranged such that, when stably connected to microplate 23, at least some of reaction sites 125 are aligned with, and/or connectable with at least a portion of wells 127 on microplate 123. In FIG. 3C, another embodiment is shown where chips 130, 131, . . . 132, are constructed and arranged such that the chips can be stably connected to each other. In some cases, chips 130, 131, . . . 132 are constructed and arranged such that, when stably connected to each other, reaction site 135 of chip 130 is aligned with one or more other reaction sites on other chips, for example, with reaction site 136 in chip 131, and/or reaction site 137 in chip 132.

The disclosed chips can be substantially liquid-tight in one set of embodiments. As used herein, a “substantially liquid-tight chip” or a “substantially liquid-tight reactor” is a chip or reactor, respectively, that is constructed and arranged, such that, when the chip or reactor is filled with a liquid such as water, the liquid is able to enter or leave the chip or reactor solely through defined inlets and/or outlets of the chip or reactor, regardless of the orientation of the chip or reactor, when the chip is assembled for use. In this set of embodiments, the chip is constructed and arranged such that when the chip or reactor is filled with water and the inlets and/or outlets sealed, the chip or reactor has an evaporation rate of less than about 100 microliters per day, less than about 50 microliters per day, or less than about 20 microliters per day. In certain cases, a chip or reactor will exhibit an unmeasurable, non-zero amount of evaporation of water per day. The substantially liquid-tight chip or reactor can have a zero evaporation rate of water in other cases.

Disclosed chips may be fabricated using any suitable manufacturing technique for producing a chip having one or more reactors, each having one or multiple reaction sites, and the chip can be constructed out of any material or combination of materials able to support a fluidic network necessary to supply and define at least one reaction site. Non-limiting examples of microfabrication processes that may be utilized for fabricating certain chips according to certain embodiments include wet etching, chemical vapor deposition, deep reactive ion etching, anodic bonding, injection molding, hot pressing, and LIGA. For example, the chip may be fabricated by etching or molding silicon or other substrates, for example, via standard lithographic techniques. The chip may also be fabricated using microassembly or micromachining methods, for example, stereolithography, laser chemical three-dimensional writing methods, modular assembly methods, replica molding techniques, injection molding techniques, milling techniques, and the like as are known by those of ordinary skill in the art. The chip may also be fabricated by patterning multiple layers on a substrate (which may be the same or different), for example, as further described below, or by using various known rapid prototyping or masking techniques. Examples of materials that can be used to form chips include polymers, silicones, glasses, metals, ceramics, inorganic materials, and/or a combination of these. The materials may be opaque, semi-opaque translucent, or transparent, and may be gas permeable, semi-permeable or gas impermeable. In some cases, the chip may be formed out of a material that can be etched to produce a reactor, reaction site and/or channel. For example, the chip may comprise an inorganic material such as a semiconductor, fused silica, quartz, or a metal. The semiconductor material may be, for example, but not limited to, silicon, silicon nitride, gallium arsenide, indium arsenide, gallium phosphide, indium phosphide, gallium nitride, indium nitride, other Group III/V compounds, Group II/VI compounds, Group III/V compounds, Group IV compounds, and the like, for example, compounds having three or more elements. The semiconductor material may also be formed out of combination of these and/or other semiconductor materials known in the art. In some cases, the semiconductor material may be etched, for example, via known processes such as lithography. In certain embodiments, the semiconductor material may have the from of a wafer, for example, as is commonly produced by the semiconductor industry.

In some embodiments, a chip as disclosed herein may be formed from or include a polymer, such as, but not limited to, polyacrylate, polymethacrylate, polycarbonate, polystyrene, polyethylene, polypropylene, polyvinylchloride, polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above may also be used. The chip may also be formed from composite materials, for example, a composite of a polymer and a semiconductor material.

In some embodiments, the chip, or at least a portion thereof, is rigid, such that the chip is sufficiently sturdy in order to be handled by commercially-available microplate-handling equipment, and/or such that the chip does not become deformed after routine use. Those of ordinary skill in the art are able to select materials or a combination of materials for chip construction that meet this specification, while meeting other specifications for use for which a particular chip is intended. In other embodiments, however, the chip may be semi-rigid or flexible.

In certain embodiments, the chip may include a sterilizable material. For example, the chip may be sterilizable in some fashion to kill or otherwise deactivate biological cells (e.g., bacteria), viruses, etc. therein, before the chip is used or re-used. For instance, the chip may be sterilized with chemicals, radiated (for example, with ultraviolet light and/or ionizing radiation), heat-treated, or the like. Appropriate sterilization techniques and protocols are known to those of ordinary skill in the art. For example, in one embodiment, the chip is autoclavable, i.e., the chip is constructed and arranged out of materials able to withstand commonly-used autoclaving conditions (e.g., exposure to temperatures greater than about 100° C. or about 120° C., often at elevated pressures, such as pressures of at least one atmosphere), such that the chip, after sterilization, does not substantially deform or otherwise become unusable. Other examples of sterilization techniques include exposure to ozone, alcohol, phenolics, halogens, heavy metals (e.g., silver nitrate), detergents, quatanary ammonium components, ethylene oxide, CO₂, aldehydes, etc. In another embodiment, the chip is able to withstand ionizing radiation, for example, short wavelength, high-intensity radiation, such as gamma rays, electron-beams, or X-rays. In some cases, ionizing radiation may be produced from a nuclear reaction, e.g., from the decay of ⁶⁰Co or ¹³⁷Cs.

In one set of embodiments, at least a portion of the chip may be fabricated without the use of adhesive materials. For example, at least two components of a chip (e.g., two layers of the chip if the chip is a multi-layered structure, a layer or substrate of the chip and a membrane, two membranes, an article of the chip and a component of a microfluidic system, etc.) may be fastened together without the use of an adhesive material. For example, the components may be connected by using methods such as heat sealing, sonic welding, via application of a pressure-sensitive material, and the like. In one embodiment, the components may be held in place mechanically. For example, screws, posts, cantilevers, matching indentations, etc. may be used to mechanically hold the chip (or a portion thereof) together. In other embodiments, the two components of the chip may be joined together using techniques such as, but not limited to, heat-sealing methods (e.g., or more components of the chip may be heated to a temperature greater than the glass transition temperature or the melting temperature of the component before being joined to other components), or sonic welding techniques (e.g., vibration energy such as sound energy may be applied to one or more components of the chip, allowing the components to at least partially liquefy or soften).

In certain embodiments, two components of the chip may be fastened via a heat-sealing method. For example, one or more components of the chip may be heated to a temperature greater than the glass transition temperature or the melting temperature of the component (i.e., temperatures at which the component softens or begins to liquefy). The components can be placed in contact with each other and allowed to cool to below the glass transition temperature or the melting temperature, thus allowing the components to become fastened together.

In certain embodiments, the two components can be fastened via sonic welding techniques. As one example, vibration energy (e.g., sound energy) may be applied to one or more components of the chip. The applied vibration energy causes the component(s), or at least a portion of the component(s), to at least partially liquefy or soften. The components can then be placed together. The vibration energy may then be stopped, thus allowing the components to become fastened together. In some cases, the components may be designed such that the vibration energy is able to be concentrated into certain regions of the component (an “energy director” region), such that only the energy director region of the component is able to liquefy under the influence of the vibration energy. For example, when vibration energy is applied to a component, a substantial fraction of the energy can be concentrated in an energy director region, allowing at least a portion of the energy director to soften or liquefy. The softened and/or liquefied region may then be connected to other components of the chip and allowed to harden, thus allowing two components of the chip to be fastened together.

In another set of embodiments, two or more components of the chip may be joined using an adhesive material. As used herein, an “adhesive material” is given its ordinary meaning as used in the art, i.e., an auxiliary material able to fasten or join two other materials together. Non-limiting examples of adhesive materials suitable for use with the invention include silicone adhesives such as pressure-sensitive silicone adhesives, neoprene-based adhesives, and latex-based adhesives. The adhesive may be applied to one or more components of the chip using any suitable method, for example, by applying the adhesive to a component of the chip as a liquid or as a semi-solid material such as a viscoelastic solid. For example, in certain embodiments, the adhesive may be applied to the component(s) using transfer tape (e.g., a tape having adhesive material attached thereto, such that, when the tape is applied to the component, the adhesive, or at least a portion of the adhesive, remains attached to the component when the tape is removed from the component). In one set of embodiments, the adhesive may be a pressure-sensitive adhesive, i.e., the material is not normally or substantially adhesive, but becomes adhesive and/or increases its adhesive strength under the influence of pressure, for example, a pressure greater than about 6 atm or about 13 atm (about 100 psi or about 200 psi). Non-limiting examples of pressure-sensitive adhesives include AR Clad 7876 (available from Adhesives Research, Inc., Glen Rock, Pa.) and Trans-Sil Silicone PSA NT-1001 (available from Dielectric Polymers, Holyoke, Mass.)

In certain embodiments, the adhesive may be applied to at least a component of the chip using a solvent-bonding system. In a solvent-bonding system, one or more components of the chip are placed in an environment rich in solvent vapor, i.e., the environment that the component(s) is placed in is saturated or supersaturated with a solvent, such that the solvent is able to condense onto the component(s) placed within the environment under suitable conditions (e.g., when the pressure and/or the temperature is lowered). The components can then be contacted together within the environment and allowed to fasten together, for example, when the environment (including solvent) is removed. As one specific example, two polycarbonate components of a chip of the invention may be fastened together in a methylene chloride environment. For example, a thin layer of a solvent, i.e. methylene chloride or the like, may be applied to a surface.

The two surfaces to be joined may then be pressed and/or clamped together under pressure to ensure bonding.

In some embodiments, the chip may be constructed and arranged such that one or more reaction sites can be defined, at least in part, by two or more components fastened together as previously described (i.e., with or without an adhesive). In some cases, a reaction site may be free of any adhesive material adjacent to or otherwise in contact with one or more surfaces defining the reaction site, and this can be advantageous, for instance, when an adhesive might otherwise leach into fluid at the reaction site. Of course, an adhesive may be used elsewhere in the chip, for example, in other reaction sites. Similarly, in certain cases, a reaction site may be constructed using adhesive materials, such that at least a portion of the adhesive material used to construct the reaction site remains within the chip such that it is adjacent to or otherwise remains in contact with one or more surfaces defining the reaction site. Of course, other components of the chip may be constructed without the use of adhesive materials, as previously discussed.

Referring now to FIG. 2, one example of a microfluidic chip 40 is shown. Chip 40 includes four general units, including a mixing unit 42, heating/dispersion unit 44, reaction site 46, and separation unit 48. One or more sensors, processors, and/or actuators (not shown) can optionally be included in sensing or actuating communication with the chip, respectively. “Sensing communication” and “actuating communication,” as used herein, means that a sensor or actuator, respectively, is positioned anywhere in association with the chip such that the environment of the reaction site and/or the content of the reaction site can be determined and/or controlled. A sensor or actuator can be included within the chip, for example embedded within or integrally connected to the reaction site, positioned within or on the chip, or positioned remotely from the chip but with physical, electrical, and/or optical connection with the reaction site so as to be able to sense or actuate a factor within the reaction site. For example, a sensor may be free of any physical connection with a chip, but may be positioned so as to detect the results of interaction of electromagnetic radiation, such as infrared, ultraviolet, or visible light, which has been directed toward a reaction site and has passed through the site or has been reflected or diffracted by the site. As another example, a sensor may be positioned on or within a chip, and may sense activity at a reaction site by being connected optically to the reaction site via a waveguide. The chip can be similarly directly or indirectly connected to a network or a control system for overall control of detection and actuation. Sensing and actuating communication can also be provided where the reaction site is in communication with a sensor or actuator fluidly, optically or visually, thermally, pneumatically, electronically, or the like, so as to be able to sense a condition of the reaction site and/or the content of the site. As one example, the sensor may be positioned downstream of one of the outlets, or behind a membrane or a transparent cover separating the reaction site from the sensor. Additional discussion of sensing and actuating arrangements is provided below.

FIG. 4 illustrates another embodiment of a chip device as presently disclosed. FIG. 4A illustrates a top view and FIG. 4B illustrates a side view of chip 105. In this embodiment, chip 105 is composed of three layers of material, namely, top layer 100 (which is transparent in the embodiment illustrated), middle layer 115, and lower layer 110. Of course, in other embodiments, chip 105 may have more or fewer layers of material (e.g., including only 1 layer), depending on the specific application. In the embodiment shown in FIG. 4, middle layer 115 has one or more void spaces 112, defining a plurality of predetermined reaction sites as discussed below. One or more channels 116, 117 may also be defined within middle layer 115, in fluid communication with reaction site 112. In some cases, one or more ports 114, 118 may allow external access to the channels, for example through upper layer 100.

Upper layer 100 may cover or at least partially cover middle layer 115, thereby in part defining reaction site(s) 112. In some cases, upper layer 100 may be permeable to a gas or liquid, for example, in cases where a gas or liquid agent is allowed to permeate or penetrate through upper layer 100. For instance, upper layer 100 may be formed from a polymer such as a poly(acetylene) humidity control material as described above, etc., which may be thin enough to allow detectable or measurable gaseous transport therethrough. In some cases, gaseous transport through upper layer 100 may be possible, while the transport of a liquid through upper layer 100 is not generally possible within a reasonable time frame. In certain cases, upper layer 100 may also be substantially transparent or translucent, for example, in embodiments where light is used to initiate a reaction or activate a process (e.g., within the reaction site). In some cases, upper layer 100 may be formed from a polymer that allows a gaseous pH-altering agent to permeate across. In certain instances, upper layer 100 may be formed of a material that is self-sealing, i.e., the material may be penetrated by a solid object but generally regains its shape after such penetration. For example, upper layer 100 may be formed of an elastomeric material which may be penetrated by a mechanical device such as a needle, but which sealingly closes once the needle or other mechanical device is withdrawn.

Middle layer 115 may include four void spaces in the embodiment illustrated in FIG. 4. Of course, in other embodiments, more or fewer void spaces may be present within middle layer 115. In the embodiment illustrated in FIG. 4, void space in middle layer 115, along with upper layer 100 and lower layer 110, may define reaction site 112. In the embodiment of FIG. 4, there are four reaction sites 112, which are substantially identical; however, in other embodiments, more or fewer predetermined reaction sites may exist, and the reaction sites may each be the same or different. In the embodiment shown, each void space is substantially identical and has two fluid channels 116, 117 in communication with the void space. Of course, in other embodiments, there may be more or fewer channels running throughout the chip. In the embodiment of FIG. 4, fluid channel 116 is connected to port 118 in layer 115, and fluid channel 117 is connected to port 114 in layer 115; in other embodiments, of course, fluid channels 116 and 118 may fluidly connect one or more reaction sites to each other, to one or more fluid ports, and/or to one or more other components within chip 105. Ports 114 and/or 118 may be used to introduce or withdraw fluids or other substances from the chip in some cases. In some embodiments, reaction site 112 and/or one or more fluidic channels may be defined, for example, in one or more layers of the chip, for example, solely within one layer, at a junction between two layers, in a void space that spans three layers, etc.

Ports 114 and 118 may be in fluid communication with one or more reaction site(s) 112. Ports 114 and 118 may be accessible, in some cases, by inserting a needle or other mechanical device through upper layer 100. For example, in some cases, upper layer 100 may be penetrated, or a space in upper layer 100 may permit external access to ports 114 and/or 118. In some cases, upper layer 100 may be composed of a flexible or elastomeric material, which may be self-sealing in some cases. In certain instances, upper layer 100 may have a passage formed therein that allows direct or indirect access to ports 114 and/or 118, or ports 114 and/or 118 may be formed in upper layer 100 and connected to channels 116 and 117 through channels defined within layer 100.

Lower layer 110 forms the bottom of chip 105 illustrated in FIG. 4. As previously described, parts of lower layer 110 in part may define reaction site 112 in certain instances. In some cases, lower layer 110 may be formed of a relatively hard or rigid material, which may give relatively rigid structural support to chip 105. Of course, in other embodiments, lower layer 110 may be formed of a flexible or elastomeric material (i.e., non-rigid). In some cases, lower layer 110 may contain one or more channels defined therein and/or one or more ports defined therein. In some cases, material defining a boundary of the reaction site, such as lower layer 110 (or upper layer 100), may contain salts and/or other materials, for example, in cases where the materials are reacted in some fashion to produce an agent that is allowed to be transported to or proximate reaction site 112. The agent may be any agent as previously discussed, for instance, a gas, a liquid, an acid, a base, a tracer compound, a small molecule (e.g., a molecule with a molecular weight of less than about 1000 Da-1500 Da), a drug, a protein, or the like, and transport may occur by any suitable mechanism, for example, diffusion (natural or facilitated) or percolation. In one embodiment, the agent is produced by a thermal decomposition reaction that may be externally initiated, for example, using electric current or light (e.g., with a laser). In certain other cases, material defining a boundary of the reaction site, such as lower layer 110 or upper layer 100, may contain one or more reservoirs of agents that are not in fluidic contact with reaction site 112, but where the agents may be transported to or proximate the reaction site, for example, by creating at least one fluidic connection between a reservoir and a reaction site. The transport may be externally controlled or driven in some cases, e.g., using an electric or magnetic field to direct fluid movement. Of course, in still other cases, lower layer 110 and/or upper layer 100 may not contain any agents or other reservoirs.

It should be understood that the chips and reactors disclosed herein may have a wide variety of different configurations. For example, a chip may be formed from a single material, or the chip may contain more than one type of reactor, reservoir and/or agent. In some cases, a chip may contain more than one system able to alter one or more environmental factor(s) within one or more reaction sites within the chip. For example, the chip may contain a sealed reservoir and an upper layer that a non-pH-neutral gas is able to permeate across.

The presently disclosed chips can be constructed and arranged so as to be able to detect or determine one or more environmental conditions associated with a reaction site of the reactor, for example, using a sensor. In some cases, each reaction site may be independently determined. Detection of the environmental condition may occur, for example, by means of a sensor which may be positioned within the reaction site, or positioned proximate the reaction site, i.e., positioned such that the sensor is in communication with the reaction site in some manner. In some cases, such detection may occur in real-time. The sensor may be, for example, a pH sensor, an optical sensor, an oxygen sensor, a sensor able to detect the concentration of a substance, or the like. Other examples of sensors are further described below. The sensor may be embedded and integrally connected with the chip (e.g., within a component defining at least a portion of the reaction site a channel in fluidic communication with the reaction site, etc.), or separate from the chip in some cases (e.g., within sensing communication). Also, the sensor may be integrally connected to or separate from the reaction site in certain embodiments.

As used herein, an “environmental factor” or an “environmental condition” is a detectable and/or measurable condition (e.g., by a sensor) of the environment within and/or associated with a reaction site, such as the temperature or pressure. The factor or condition may be detected and/or measured within the reaction site, and/or at a location proximate to the reaction site (e.g., upstream or downstream of the reaction site) such that the environmental condition within the reaction site is known and/or controlled. For example, the environmental factor may be the concentration of a gas or a dissolved gas within the reaction site or associated with the reaction site (for example, upstream or downstream of the reaction site, separated from the reaction site by a membrane, etc.). The gas may be, for example, oxygen, nitrogen, water (i.e., the relative humidity), CO₂, or the like. The environmental factor may also be a concentration of a substance in some cases. For example, the environmental factor may be an aggregate quantity, such as molarity, osmolarity, salinity, total ion concentration, pH, color, optical density, or the like. The concentration may also be the concentration of one or more compounds present within the reaction site, for example, an ion concentration such as sodium, potassium, calcium, iron or chloride ions; or a concentration of a biologically active compound, such as a protein, a lipid, or a carbohydrate source (e.g., a sugar) such as glucose, glutamine, pyruvate, apatite, an amino acid or an oligopeptide, a vitamin, a hormone, an enzyme, a protein, a growth factor, a serum, or the like. In some embodiments, the substance within the reaction site may include one or more metabolic indicators, for example, as would be found in media, or as produced as a waste products from cells. If cells are present, the sensor may also be a sensor for determining all viability, cell density, cell motility, cell differentiation, cell production (e.g., of proteins, lipids, small molecules, drugs, etc.), etc.

The environmental factor may also be a fluid property of a fluid within the reaction site, such as the pressure, the viscosity, the turbidity, the shear rate, the degree of agitation, or the flowrate of the fluid. The fluid may be, for instance, a liquid or a gas. In one set of embodiments, the environmental factor is an electrical state, for example, the charge, current, voltage, electric field strength, or resistivity or conductivity of the fluid or another substance within the reaction site. In one set of embodiments, the environmental condition is temperature or pressure. In certain embodiments, the sensor may be a ratiometric sensor, i.e., a sensor able to determine a difference or ratio between two (or more) signals, e.g., a measurement and a control signal, two measurements, etc.

Non-limiting examples of sensors useful or potentially useful in the context of certain disclosed devices include dye-based detection systems, affinity-based detection systems, microfabricated gravimetric analyzers, CCD cameras, optical detectors, optical microscopy systems, electrical systems, thermocouples and thermistors, pressure sensors, etc. Those of ordinary skill in the art will be able to identify other suitable sensors. For example, in one set of embodiments, the chip may contain a sensor comprising one or more detectable chemicals responsive to one or more environmental factors, for example, a dye (or a combination of dyes), a fluorescent molecule, etc. One or more dyes, or fluorescent or chromogenic molecules sensitive to a specific environmental condition(s) may be chosen by those of ordinary skill in the art. Non-limiting examples of such dyes, or fluorescent or chromogenic molecules include pH-sensitive dyes such as phenol red, bromothymol blue, chlorophenol red, fluorescein, HPTS, 5(6)-carboxy-2′,7′-dimethoxyfluorescein SNARF, and phenothalein; dyes sensitive to calcium such as Fura-2 and Indo-1; dyes sensitive to chloride such as 6-methoxy-N-(3-sulfopropyl)-quinolinim and lucigenin; dyes sensitive to nitric oxide such as 4-amino-5-methylamino-2′,7′-difluorofluorescein; dyes sensitive to dissolved oxygen such as tris(4,4′-diphenyl-2,2′-bipyridine) ruthenium (II) chloride pentahydrate; dyes sensitive to dissolved CO₂; dyes sensitive to fatty acids, such as BODIPY 530-labeled glycerophosphoethanolamine; dyes sensitive to proteins such as 4-amino-4′-benzamidostilbene-2-2′-disulfonic acid (sensitive to serum albumin), X-Gal or NBT/BCIP (sensitive to certain enzymes), Tb³⁺ from TbCl₃ (sensitive to certain calcium-binding proteins), BODIPY FL phallacidin (sensitive to actin), or BOCILLIN FL (sensitive to certain penicillin-binding proteins); dyes sensitive to concentration of glucose, lactose or other components, or dyes sensitive to proteases, lactates or other metabolic byproducts, dyes sensitive to proteins, antibodies, or other cellular products, such as calcein AM, ethidium bromide, or resazurin (sensitive to viability).

In one embodiment, the dye or fluorescent molecule may be immobilized within one or more walls within the chip, e.g., within one or more walls defining the reaction site. In another embodiment, the dye or fluorescent molecule may be immobilized within a gel positioned within the chip, for example, in fluid communication with the reaction site. In yet another embodiment, the dye or fluorescent molecule may be dissolved in a media, for example, that is passed through the reaction site. The dye or fluorescent molecule may have a response generally proportional to a value of one or more environmental factors and/or other variable(s) of interest. The response may be measured, e.g., as a fluorescent signal, an absorbance signal, a wavelength or frequency, etc. A reactor and/or reaction site within a chip may be coupled to a light delivery and/or other light interacting component(s). For example, the light-interacting component may include a detection system where light (e.g., having a predetermined wavelength) arising from a dye, a fluorescent molecule, etc., may be detected and/or measured.

The sensor can include a colorimetric detection system in some cases, which may be external to the chip, or microfabricated into the chip in certain cases. In one embodiment, the colorimetric detection system can be external to the chip, but optically coupled to the reaction site, for example, using fiber optics or other light-interacting components that may be embedded in the chip (e.g., such as those described below). As an example of a calorimetric detection system, if a dye or a fluorescent molecule is used, the colorimetric detection system may be able to detect a change or shift in the frequency and/or intensity of the dye or fluorescent molecule in response to a change or shift in one or more environmental factors within a reaction site. As a specific example, Ocean Optics Inc. (Dunedin F.O.) provides fiber optic probes and spectrometers for the measurement of pH and dissolved oxygen concentration.

In certain embodiments, any one or more of the above-described chips may be constructed and arranged such that the chip, or a portion thereof, such as one or more reaction sites, is able to respond to a change in an environmental condition within or associated with a reaction site, for example, by use of a control system. In some cases, each reaction site within the chip may be independently controlled in some fashion. As used herein, a “control system” is a system able to detect and/or measure one or more environmental factors within or associated with the reaction site, and cause a response or a change in the environmental conditions within or associated with the reaction site (for instance, to maintain an environmental condition at a certain value). In some cases, the control system may control the environmental factor in real time. The response produced by the control system may be based on the environmental factor in certain cases. An “active” control system, as used herein, is a system able to cause a change in an environmental factor associated with a reaction site as a direct response to a measurement of the environmental condition. The active control system may provide an agent that can be delivered, or released from the reaction, where the agent is controlled in response to a sensor able to determine a condition associated with the reaction site. A “passive” control system, as used herein, is a system able to maintain or cause a change in an environmental condition of the reaction site without requiring a measurement of an environmental factor. The passive control system may control the environmental factor within the reaction site, but not necessarily in response to a sensor or a measurement. The passive control system may allow an agent to enter or exit the reaction site without active control. For example, a passive control system may include an oxygen membrane, a water-permeable membrane, and/or a humidity control membrane as described above, where the membrane can maintain the oxygen and/or the water vapor content within the reaction site, for instance, within certain predetermined limits. The control system may be able to control one or more conditions within or associated with the reaction site for any length of time, for example, 1 day, 1 week, 30 days, 60 days, 90 days, 1 year, or indefinitely in some cases.

The control system can include a number of control elements, for example, a sensor operatively connected to an actuator, and optionally to a processor. One or more of the components of the control system may be integrally connected to the chip containing the reaction site, or separate from the chip. In some cases, the control system includes components that are integral to the chip and other components that are separate from the chip. The components may be within or proximate to the reaction site (e.g., upstream or downstream of the reaction site, etc.). Of course, in some embodiments, the control system may include more than one sensor, processor, and/or actuator, depending on the application and the environmental factor(s) to be detected, measured, and/or controlled. One example of a control system is depicted in FIG. 5, in which an environmental condition 50 within chip 105, detected by a sensor 52, is transduced into a signal 51 that is transmitted to processor 54 for suitable processing. Processor 54 then produces a signal 53, which is sent to actuator 56 where the signal is converted into a response 60. In some embodiments, the control system may be able to produce a very rapid change in the environmental factor in response to a stimulus or a change in stimulus (for example, a detectable change in an environmental factor such as temperature or pH in a time of less than 5 s, less than 1 s, less than 100 ms, less than 10 ms, or less than 1 ms).

As used herein, a “processor” or a “microprocessor” is any component or device able to receive a signal from one or more sensors, store the signal, and/or convert the signal into one or more responses for one or more actuators, for example, by using a mathematical formula or an electronic or computational circuit. In one embodiment, the processor may be an expert system. The signal may be any suitable signal indicative of the environmental factor determined by the sensor, for example a pneumatic signal, an electronic signal, an optical signal, a mechanical signal, etc. Processor 54 may be any device suitable for determining a response to the signal, for example, a mechanical device or an electronic device such as an integrated circuit. The processor may be embedded and integrally connected with the reaction site or chip, or separate from the reaction site or chip, depending on the application. In one embodiment, the processor is programmed with a process control algorithm, which can, for example, take an incoming signal from a sensor and convert the signal into a suitable output for an actuator. Any suitable algorithm(s) may be used within processor 54, for example, a PID control system, a feedback or feedforward system, a fuzzy logic control system etc. The processor may be programmed or otherwise designed to control an environmental condition within the reaction site, for example, by manipulation of an actuator.

For example, in certain embodiments, processor 54 is able to maintain one or more environmental conditions (e.g., temperature or pressure) at a constant, predetermined level within a predetermined reaction site of a chip, for example, to facilitate a chemical reaction therein. In certain embodiments, processor 54 is able to alter one or more environmental conditions within one or more predetermined reaction sites of a chip according to a predetermined pattern, or in response to a specific condition; for example, the processor may cause the actuator to raise the pH within a predetermined reaction site at a certain rate, the processor may cause the actuator to alter the pH of a predetermined reaction site once a specific temperature or other environmental condition has been reached, or the processor may cause the actuator to allow or prevent, or increase or decrease, the flow of a substance or an agent into a predetermined reaction site. In some embodiments, processor 54 is able to control several environmental conditions within a predetermined reaction site, for example, at least two, three, four, five, six, seven or more conditions, preferably simultaneously or nearly simultaneously depending on the application and the degree of control that is desired. For example, processor 54 may be in communication with one or more sensors and/or one or more actuators.

In certain embodiments, processor 54 may be programmed or designed to maintain one or more environmental conditions within one or more reaction sites. For example, processor 54 may be programmed or designed to maintain one or more environmental conditions within three reaction sites, within seven reaction sites, within ten reaction sites, etc. For example, where there are a plurality of reaction sites, one subset of reactions site may be held at one temperature, while a different subset of reaction sites may be held at a different temperature. As another example, one subset of reaction sites may have a first compound added thereto, while a second subset reaction sites may have a different compound added thereto. Combinations of subsets may also be used, for example, different subsets having different chemicals, temperatures, or the like. Thus, many different environmental conditions may be simultaneously controlled at different values within one chip. In some cases, the pattern of control and monitoring of the reaction sites may be altered in time, i.e., during an experiment. Thus, for instance, two reaction sites that were monitored and/or controlled simultaneously at a first point in time may be separately monitored and/or controlled at a second point in time. The control and monitoring may be preset, automated, or manually determined.

In one set of embodiments, processor 54 may be programmed or designed to maintain conditions suitable for supporting the metabolism or growth of a cell (e.g., a bacterial or a mammalian cell). For example, processor 54 may be able to control one or more of the temperature, relative humidity, pressure, oxygen concentration, CO₂ concentration, serum concentration, nutrient concentration, shear rate, or the pH within the reaction sites of the chip. Other environmental factors suitable for supporting cell growth are described below or previously.

As used herein, an “actuator” is a device able to affect the environment within or proximate to one or more reaction sites, or in an inlet or outlet in fluid communication with one or more reaction sites (e.g., as in channels 116 and 117 in FIG. 4A). The actuator may be separate from, or integrally connected to the reaction site or chip. For example, in some embodiments, the actuator may include a valve or a pump (including microvalves and micropumps) able to control, alter, and/or prevent the flow of a substance or agent into or out of the reaction site, for example, a chemical solution, a buffering solution (e.g., a pH buffering solution), a gas such as CO₂ or O₂, a nutrient solution, a saline solution, an acid, a base, a solution containing a carbon source, a nitrogen source, an inhibitor, a promoter, a hormone, a growth factor, an inducer, etc. The substance to be transported will depend on the specific application. In some cases, the pump may be external of the chip. As one example, the actuator may selectively open a valve that allows CO₂ or O₂ to enter the reaction site. In other cases, the pump may be internal of the chip. For example, the pump may be a piezoelectric pump or a mechanically-activated pump (e.g., activated by pressure, electrical stimulation, etc.). In one embodiment, the pump is activated by producing a gas within the chip, which may cause fluid flow within the chip; as examples, the gas may be produced by directing light such as laser light at a reactant to start a gas-producing reaction, or the gas may be produced by applying an electric current to a reactant (for instance, an electric current may be applied to water to produce gas). As another example, the actuator may include a pumping system that can create a fluid connection with a reaction site as necessary. In one particular example, a chip having a gas-permeable surface may be placed in an incubator or other enclosed environment, and the atmosphere within the incubator or other environment may be controlled, thereby controlling the environmental conditions within the reaction sites.

As yet another example, the actuator may include a heating element or a cooling element, such as a heat exchanger (e.g., as shown in FIG. 2), a resistive heater or a Peltier cooler. In other embodiments, the actuator may include an electrical system, such as an electrical system that maintains a steady current, or a steady electric field gradient within the reaction site. In yet another example where at least two fluid streams enter or leave a reaction site, the actuator may include a valve or a pump that is able to control the ratio of flowrates between the two fluid streams. For instance, the actuator, in response to a signal, may act to increase an inlet flowrate and decrease an outlet flowrate within the reaction site.

In one set of embodiments, the actuator may include an energy source, such as an electromagnetic energy source, a heat source, a mechanical energy source, or an ultrasound source. In some embodiments, the electromagnetic radiation may have wavelengths or frequencies in the optical or visual range (e.g., having a wavelength of between about 400 nm and about 700 nm), infrared wavelengths (e.g., having a wavelength of between about 300 nm and 700 nm), ultraviolet wavelengths (e.g., having a wavelength of between about 400 nm and about 10 nm), or the like. In some cases, the light may cover a range of frequencies, for example, between about 350 nm and about 1000 nm, between about 300 nm and about 500 nm, between about 500 nm and about 1 nm, between about 400 nm and about 700 nm, between about 600 nm and about 1000 nm, or between about 500 nm and about 50 nm. In other cases, the light may be monochromatic (i.e., having a single frequency or a narrow frequency distribution), for example, a frequency that is commonly produced by commercial lasers, or a frequency that a fluorescent agent is excited at. For example, the frequency may be a frequency that is centered around 366 nm, 405 nm, 436 nm, 546 nm, 578 nm, 457 nm, 488 nm, 514 nm, 532 nm, 543 nm, 594 nm, 633 nm, 568 nm, or 647 nm. The monochromatic beam of light may have a narrow distribution of frequencies. For example, 90% or 95% of the frequencies may be within ±5 nm or ±3 nm of the average frequency. In certain cases, the light may be polarized (e.g., linearly or circularly), or more than one wavelength of light may be used, for example, serially or simultaneously. In some embodiments, a light-interacting component may alter the wavelength of light within the device.

In certain embodiments, the actuator may be constructed and arranged to selectively kill or deactivate specific cells or types of cells, preferably without affecting nearby or adjacent cells. For example, the actuator may include an energy source directed substantially at the reaction site, or at an inlet or outlet in fluid communication with the reaction site; on detection of a specific cell or cell type by the sensor, the actuator may target the cell, for example, by directing energy at the cell, killing the cell or otherwise deactivating it in some fashion (e.g., by damaging its DNA enough to prevent replication). The energy targeted towards the cell may be any energy able to deactivate the cell, for example, electromagnetic or ionizing radiation, ultrasound, or heat energy.

In certain embodiments, a chip may be constructed and arranged to control an environmental factor associated with a reaction site by transporting an agent able to affect the environmental factor, or a precursor of an agent that is able to affect the factor, into or proximate the reaction site (i.e., such that it affects the environmental factor within the reaction site). Control of the delivery of the agent (or precursor) to the reaction site, in certain instances, may be used to control the environmental factor.

In certain embodiments, an environmental factor within or associated with the reaction site may be altered and/or controlled without directly contacting the reaction site to an agent, e.g., an external or unsterilized agent, such as a liquid or a gas. For example, the reaction site may contain a biological specimen or a substance for use in a biological setting where sterility and/or isolation is required; or the reaction site may contain a reaction that is sensitive to, e.g., liquids or pH changes, for example, a water-sensitive reaction which must be performed in a non-humid environment, where direct contact between the agent and, the reaction site would present difficulties.

In certain embodiments, as discussed above, the chip may be constructed and arranged to allow an agent to permeate or diffuse into the reaction site. For instance, the reaction site may be defined, at least in part, by a component such as a wall or a layer of the chip, through which an agent is able to permeate. The agent may be able to alter and/or control one or more of the environmental factors within or humidity control membrane (e.g., as described above) or a semipermeable membrane (e.g., with respect to the agent) that the agent is able to permeate across. In some cases, the component may be chemically or physically inert with respect to the agent. In certain instances, a flow of agent may occur on one side of the component. In some embodiments, the flow of agent on one side of the component may occur along a meandering or non-straight pathway, for example, to increase the time of contact between the agent and the component. For example, in FIG. 2, if compartment 20 is separated from compartment 42 by a membrane (not shown) through which an agent is able to permeate, a flow of agent may occur along serpentine path 281.

In certain embodiments, a chemical agent generated elsewhere within the chip may be allowed to interact with the reaction site(s) to control the environmental factor(s) therein, or one or more fluidic pathways may be created (e.g., opened) within the chip that allows an agent stored within the chip or external the chip to come into contact with the reaction site or otherwise affect the reaction site. The agent may be any agent able to alter and/or control one or more environmental factors within the reaction site. For instance, the agent may be a non-pH-neutral composition or a pH-altering agent as previously described. As an example, in FIG. 4A, chip 105 may be constructed to allow an agent to permeate and/or diffuse into the reaction site. For instance, the reaction site may include a component such as a humidity control material (e.g., as previously described), a wall (e.g., a wall of predetermined reaction site 112) or one or more layers of the chip (e.g., upper layer 100), through which an agent is able to permeate through to affect the reaction site. As another example, the component that the agent is able to penetrate in some fashion may include or be defined by a membrane, such as an osmotic membrane or a semipermeable membrane (e.g., semipermeable with respect to the agent) that the agent is able to permeate across. In some cases, the component may be chemically or physically inert with respect to the agent; for instance, the component may allow an acidic or an alkaline compound to permeate across to the reaction site without substantially damaging or altering the component. In certain instances, a flow of agent may occur on one side of the component. In some embodiments, the flow of agent on one side of the component may occur along a meandering or non-straight pathway, for example, to increase the time of contact between the agent and the component.

For instance, in certain embodiments, a chip may have a predetermined reaction site and a permeable upper layer. In this example, a dispensing unit is positioned proximate the reaction site such that the dispensing unit is able to produce an agent able to permeate towards and interact with a reaction site within a desired time frame, for example, within a few seconds or tens of seconds, minutes, or hours, depending on the application. The dispensing unit may also be connected to one or more chemical sources, for example, one or more sources of gases and/or pH-altering agents. As examples, the sources may be an acid source and an alkaline source, the sources may each be acid sources or alkaline sources, a source may be a source of cell media, or a source of glucose or saline, etc.

As one example, if the environmental factor within or associated with the reaction site is pH, then the agent may be a pH-altering agent able to be delivered or transported to or proximate the reaction site to control the pH therein. As used herein, a “pH-altering” agent is any agent able to alter the pH of the environment within or associated with the reaction site, for example, an acid, a base, or an agent able to react within or proximate the reaction site to form an acid or a base. In some embodiments, the pH-altering agent is inert relative to the reaction site, and/or other component(s) of the chip. The pH-altering agent may be able to alter the pH of the environment within or associated with the reaction site to a significant or a measurable extent, for example, by at least 0.1, 0.2, 0.3, 0.4, 0.5, 0.8, 1, 2, or 3 or more pH units, depending on the required sensitivity and the specific application. The required pH sensitivity can be readily determined by those of ordinary skill in the art. For example, a chemical process that requires a change in pH to initiate a reaction may require large pH changes, while a process to regulate the pH of the reaction site near an optimum value may require sensitivity to smaller changes in pH.

As used herein, “acid” is given its ordinary definition as used in chemistry. An acid may have a pH of less than about 6.99, less than 5, less than 4, less than 3, or less than 2 pH units, depending on the strength of the acid. Similarly, a “base,” or an “alkaline” is given its ordinary definition as used in the field of chemistry. A base or alkaline may have a pH of at least about 7.01, at least about 8, at least about 9, at least about 11, or at least about 12 pH units. A “non-neutral” or a “non-pH-neutral” composition is a composition that is either acidic or basic (i.e., the composition has a pH that is either greater than or less than 7, preferably by a significant amount, such as by at least 1 or 2 pH units). The non-pH-neutral composition may be a solid, a liquid, or a gas in some cases. As used herein, a “gaseous” acid or base is a composition that is in the gas phase, or is generally volatile (i.e., having a high vapor pressure) and easily enters the gas phase. For example, the gaseous acid or base may have a vapor pressure of at least about 300 mmHg, at least about 400 mmHg, at least about 500 mmHg, at least about 600 mmHg, or at least about 700 mmHg. Non-limiting examples of gaseous acids include acetic acid, formic acid, propionic acid, pyruvic acid, lactic acid, SO₂, CO₂, CO, NO₂, or butyric acid; non-limiting examples of gaseous bases include ammonia, phosphine, or arsine.

In some embodiments, the environmental factor within the reaction site may be altered by generating one or more agents within the chip, for example, from one or more precursors. The agent(s) may interact with, or alter in some way, an environmental factor within the reaction site. In one embodiment, the agent may be generated within the reaction site. In another embodiment, the agent may be generated elsewhere within the chip and transported to the reaction site in some fashion, for instance, fluidically. For example, the chemical agent may be produced and/or stored within a different compartment associated with or external of the chip (e.g., as in a reservoir), then transported to the reaction site, for instance, through a channel or other fluidic connection, or by allowing it to permeate or diffuse across a membrane or another component. In one embodiment, the agent may be generated in a location proximate the reaction site, e.g., the agent may be generated in a location where it can be readily transferred or transported to the reaction site, for example, within a few seconds or tens of seconds. In another embodiment, the agent may be a gas that is transported to the reaction site, for example, through a membrane, or over a barrier that prevents liquid communication between the compartment and the reaction site, while non-gaseous products may be prevented from entering the reaction site. In certain embodiments, the reaction of the precursor(s) that produces the agent may be externally initiated. For example, a light source, such as a laser, may be applied to the precursor(s), or other energy sources such as electrical current or heat may be used to initiate a reaction of the precursor(s). In yet another embodiment, a fluidic connection may be created between the compartment and the reaction site, for example, reversibly. For instance, the fluidic connection may be created by opening a valve such as a mechanical valve or a micromechanical valve, etc. separating the compartment and the reaction site.

In some cases, additional compounds may be combined with the precursor(s) to, for example, preserve the precursor(s) against decomposition or degradation, to enhance the ability of the precursor(s) to react (e.g., a catalyst or an enzyme), or to enhance the absorption of incident energy onto the precursor(s), for instance, to increase the reaction rate of the precursor(s) to form an agent. In some embodiments, a material that is able to absorb of incident electromagnetic radiation, such as a darkened or “black” material, may be added to the precursor(s), for example, to enhance the absorption of energy. Non-limiting examples of such materials include quartz, black glass, silicon, black sand, carbon black, and the like. The additional compounds may be substantially unreactive, unable to form a transportable agent (i.e., transportable through a layer or a component of the chip), or the additional compounds may not significantly interfere with the production of the agent or with control of an environmental factor associated with the reaction site.

The agent, in certain embodiments, may be produced in a reaction that is activated at a certain temperature, such as in a thermal decomposition or degradation reaction. In some cases, the reaction to produced the agent may be initiated when the precursor(s) is exposed to at least a certain temperature able to activate the reaction, for example, a temperature of at least about 200° C., 300° C., 400° C., or 500° C. The temperature necessary to activate the reaction may be produced within the precursor(s) by any suitable technique, for example, upon the exposure of light energy, heat, electrical energy (e.g., resistive heating), an exothermic chemical reaction, or the like to the precursor(s).

In some embodiments, the agent so produced may be a gas, for example, O₂, CO, CO₂, NO, NO₂, HCl, or the like. In some cases, the agent-producing reaction may produce one or more gases and/or one or more non-gaseous products. In some cases, the gaseous agent(s) may then be transported into or proximate the reaction site (for example, through a membrane or over a barrier), while non-gaseous products (such as liquids or solids) may be prevented from entering the reaction site in some fashion.

The agent, in certain cases, may be a pH-altering agent. In some cases, the pH-altering agent may be a base, such as ammonia. The base may be generated by any suitable reaction that can generate an alkaline agent, such as through a thermal decomposition reaction of an alkaline precursor salt. For example, ammonia may be generated through the thermal decomposition of an ammonium precursor salt such as ammonium nitrate, ammonium carbonate, ammonium bicarbonate, ammonium chloride, ammonium bromide, ammonium fluoride, or the like. In other cases, the pH-altering agent may be an acid, such as acetic acid or formic acid. The acid may be generated using any suitable reaction that can generate an acidic agent, such as the thermal decomposition of an acid precursor salt. For instance, acetic acid may be produced by the thermal decomposition of sodium acetate, potassium acetate, calcium acetate, lithium acetate, magnesium acetate, or the like. Similarly, formic acid may be produced by the thermal decomposition of sodium formate, potassium formate, calcium formate, lithium formate, magnesium formate, etc. In some cases, the pH-altering agent may not be an acid or a base, but be in a form that can be converted into an acid or a base within the chip or within a reaction site. For example, the pH-altering agent may react with water to form an acid or a base within the chip or reaction site. As a non-limiting example, a gas such as CO₂ may react with water to produce carbonic acid, e.g.:
CO₂+H₂O<->H₂CO₃<->H⁺+HCO₃⁻.

In yet another set of embodiments, the agent may be present in a compartment not in fluid communication with the reaction site; when exposure of the agent to the reaction site is desired in order to alter or control an environment factor therein is desired, a fluidic pathway may be created to enable the agent to enter into or otherwise interact with the reaction site. For example, a created fluidic pathway may be a new pathway, i.e., a non-preexisting pathway, or a pathway created in a region that did not previously contain a fluidic pathway; or the created fluidic pathway may be created in a region that previously contained a fluidic pathway that has been altered to prevent fluidic communication. In some cases, a new pathway may be created within the chip by removing or damaging a component of the chip, such as a layer, a membrane a wall defining a reaction site or a channel in fluidic communication with the reaction site, etc. As another example, the fluidic pathway may be a closed, pre-existing fluidic pathway that can be opened and/or modified under certain conditions, for instance, a valve or a switch. In one embodiment, the compartment is a sealed compartment, e.g., a compartment without access to the external environment and/or the reaction site. In another embodiment, the compartment is accessible externally (i.e., through an inlet or an outlet), but is not in fluid communication with the reaction site.

Certain disclosed chips may include one or more fluid pathways for delivery of species or removal of species from a reaction site. In some cases, a fluidic pathway can be created in situ (after construction of the chip, during chip setup and/or during use of the chip) by permeabilizing or damaging a component separating the compartment from the reaction site (e.g., as in a wall or a membrane), or separates the compartment from a fluidic pathway in fluid communication with the reaction site. For instance, in certain embodiments of the invention, the fluidic pathway or other means for fluidic communication may be created by permeabilizing and/or damaging (reversibly or irreversibly) a component that separates the compartment containing the agent (and/or agent precursor(s)) from fluidic communication with the reaction site, or separates the compartment from a channel or other fluidic pathway in fluid communication with the reaction site, thus creating a fluidic connection between the compartment and the reaction site. For example, the component may be permeabilized by heating the component to increase the permeability of the chemical agent or by causing the component to melt or vaporize. In some cases, the permeability of the component may be enhanced by one, two, or three or more orders of magnitude. In certain cases, the permeabilization of the component may be reversible or at least partially reversible, for example, by decreasing the temperature, or introducing a non-permeabilizing agent.

The component, in some cases, may also be damaged or otherwise altered or permeabilized through a reaction, for example, a chemical or electrochemical reaction, to produce a fluidic connection with the reaction site. For example, the component may include a metal, such as gold, silver or copper, that can be electrolyzed upon the application of a suitable electrical current. As yet another example, the component may be chemically etched, for example, with a reactive species.

In certain embodiments, the component as discussed above may be mechanically altered and/or damaged, for example, by piercing the component with a microneedle to create a fluidic pathway between the compartment and the reaction site. The microneedle or other mechanical device may originate from within the chip, or externally. In one embodiment, the component may be altered on a reversible basis, for example, the component may be self-sealing and/or comprised an elastomeric substance that can be resealed.

The component may also be damaged without the use of mechanical forces or chemicals in some cases. For example, energy may be applied to the surface to damage it. In some embodiments, the component may be ablated, for example, using heat or light. If light is used, the light may be channeled through a waveguide to the surface in some cases, or light may be applied directly to the surface.

The component may include a material able to enhance the creation of the fluidic pathway in some embodiments. As examples, the enhancing material may facilitate the absorption of light or other forms of energy, or increase the chemical reaction or transport rate. For instance, in one embodiment, the component may comprise a material that is able to absorb incident electromagnetic radiation, i.e., a darkened or “black” material, such as quartz, black glass, silicon, black sand, carbon black, and the like. As other examples, the component may include a catalyst, an enzyme, or a permeation enhancer.

The presently disclosed chips may include a variety of other components. For example, a chip may include components such as a light source, a flowmeter (e.g., for measuring fluid flow of a gas or a liquid), a circuit such as an integrated circuit, a reservoir (e.g., for a solution), a micromechanical or a MEMS (“microelectromechanical system”) component, a microvalve, a micropump, or the like, for example, as further described below. The components may be fabricated on the chip using techniques such as those used in standard microfabrication, similar to those used to create semiconductors (See Madou Fundamentals of Microfabrication, CRC Press, Boca Raton, Fla. 1997; and Maluf, An Introduction of Micromechanical Systems Engineering, Artech House Boston, Mass. 2000). In some embodiments, at least one, two, three or more components are integrally connected to the chip. In certain embodiments, all of the components are integrally connected to the chip.

Other examples of components suitable for use with the invention include pylon-like obstructions placed in the flow path of a stream to enhance mixing within the chip, reactor and/or reaction site, or heating, separation, and/or dispersion units within the chip, reactor and/or reaction site. For example, if a heating unit is present, the heating unit may be a miniaturized, traditional heat exchanger.

For instance, in one set of embodiments, the presently disclosed devices may include a membrane, such as a membrane that may control humidity (e.g., as previously described) and/or be substantially transparent. If a membrane is present, it may be positioned anywhere in a reactor within a chip. In certain embodiments, the membrane is positioned such that it defines the surface of one or more reaction sites and/or divides a reaction site into two or more portions, which portions may have the same or different dimensions. For example, in FIG. 7A, membrane 410, which may be a humidity controller and/or be substantially transparent, defines a surface of reaction site 411. In FIG. 7B, membrane 410 defines the surface of reaction site 411 and a surface of reaction site 412. As another example, the membrane can be positioned such that it is in fluidic communication with one or more reaction sites of the chip. In some cases, the membrane may be positioned such that a pathway fluidly connecting a first reaction site with a second reaction site crosses the membrane. In another embodiment, the membrane can be positioned such that it is in fluidic communication with one or more reaction sites of the chip. In some cases, the membrane may be positioned such that a pathway fluidly connecting a first reaction site with a second reaction site crosses the membrane. For example, in FIGS. 7C and 7D, membrane 410 does not define surfaces of reaction sites 411 or 412, but is positioned such that at least one pathway fluidly connecting reaction site 411 with reaction site 412 crosses membrane 410.

As one example, in one embodiment, the membrane may be a porous membrane having, for example, a number-average pore size of greater than about 0.03 micrometers and less than about 5 micrometers. In other embodiments, the pore size of the membrane may be less than about 4 micrometers, less than about 3 micrometers, less than about 2 micrometers, less than about 1.5 micrometers, less than about 1.0 micrometers, less than about 0.75 micrometers, less than about 0.6 micrometers, less than about 0.5 micrometers, less than about 0.4 micrometers, less than about 0.3 micrometers, less than about 0.1 micrometers, less than about 0.07 micrometers, and in other embodiments, less than about 0.05 micrometers. In certain cases, the pores are also greater than 0.03 micrometers or greater than 0.08 micrometers. In some cases, the membrane may be chosen to prevent the passage of certain cells there through (e.g., bacterial cells, yeast cells, mammalian cells, etc.). For example, a membrane with a pore size of about 0.2 micrometers may prevent the passage of bacteria cells, and a membrane with a pore size of a bout 1 micrometer may prevent the passage of mammalian cells. In certain embodiments, a membrane may be chosen to prevent or permit the passage of certain molecules, e.g., micromolecules, having a certain size and/or charge, i.e., a charge and/or size selective membrane.

A membrane, when configured as a humidity controller, may be constructed of materials described previously. In these, as discussed previously, or other embodiments, a membrane may include polymers or other materials such as polyethylene terephthalate (PET), polysulfone, polycarbonate, acrylics such as polymethyl methacrylate, polyethylene, polypropylene, regenerated cellulose, nitrocellulose, aluminum oxide, glass, fiberglass, and the like. In certain embodiments, a membrane may also be substantially transparent, e.g., as previously described. In one embodiment, for example, a membrane is a substantially transparent polyethylene terephthalate membrane having a pore size of 2 micrometers or less, for example, a ROTRAC® capillary membrane made by Oxyphen U.S.A., Inc. (New York, N.Y.).

In one set of embodiments, a chip may include a structure adapted to facilitate the reactions or interactions that are intended to take place therein (e.g., within a reaction site). For example, where a chip is intended to function as one or more bioreactors for cell culturing, the chip may include structure(s) able to improve or promote cell growth. For instance, in some cases, a surface of a reaction site may be a surface able to promote cell binding or adhesion, or the reactor and/or reaction site within the chip may include a structure that includes a cell adhesion layer, which may include any of a wide variety of hydrophilic, cytophilic, and/or biophilic materials. As examples, the surface may be ionized, coated (e.g., with a support material) and/or micropatterned with any of a wide variety of hydrophilic, cytophilic, and/or biophilic materials, for example, materials having exposed carboxylic acid, alcohol, and/or amino groups. Examples of materials that may be suitable for a cell adhesion layer include, but are not limited to, polyfluoroorganic materials, polyester, PDMS, polycarbonate, polystyrene, and aluminum oxide. As another example, the structure may include a layer coated with a material that promotes cell adhesion, for example, an RGD peptide sequence, or the structure may be treated in such a way that it is able to promote cell adhesion, for example, the surface may be treated such that the surface becomes relatively more hydrophilic, cytophilic, and/or biophilic. In some embodiments, it may be desired to modify the surface of a cell adhesion layer, for instance with materials that promote cell adhesion, for example, by attachment, binding, soaking or other treatments. Example materials that promote cell adhesion include, but are not limited to, fibronectin, laminin, albumin or collagen. In other embodiments, for example, where certain types of bacteria or anchorage-independent cells are used, the surface may be formed out of a hydrophobic, cytophobic, and/or biophobic material, or the surface may be treated in some fashion to make it more hydrophobic, cytophobic, and/or biophobic, for example, by using aliphatic hydrocarbons and/or fluorocarbons.

In some embodiments, the chip may include a “light-interacting component,” i.e., a component that interacts with light, for example, by producing light, reacting to light, causing a change in a property of light, directing light, altering light, etc. As used herein, a “light-interacting component” is a component that interacts with light in some fashion related to chip and/or reactor function, for example, by producing light, reacting to light, causing a change in a property of light, directing light, altering light, etc., in a manner that affects a sample within a chip or reactor and/or determines something about the sample (the presence of the sample, a characteristic of the sample, etc.). In one embodiment, the component produces light, such as in a light-emitting diode (“LED”) or a laser. In another embodiment, the light-interacting component may be a component that is sensitive to light or responds to light, such as a photodetector or a photovoltaic cell. In yet another embodiment, the light-interacting component may manipulate or alter light in some fashion, for example, by focusing or collimating light, or causing light to diverge, such as in a lens, or spectrally dispersing light, such as in a diffraction grating or a prism. In another embodiment, the light-interacting component may be able to transmit or redirect the direction of light in some fashion, such as along a bent path or around a corner, for example, as in a waveguide or mirror. In yet another embodiment, the light-interacting component may alter a property of light incident on the component, such as the degree of polarization or the frequency, for example, as in a polarizer or an interferometer. Other devices, or combinations of devices, are also possible. In general, the term “light-interacting component” does not encompass components or devices that passively transmit light without significant modification, alteration, or redirection, such as air, or a plane of glass or plastic (e.g., a “window”). The term “light-interacting component” also does not generally encompass components that passively absorb essentially all incident light without a response, such as would be found in an opaque material.

In embodiments in which a light-interacting component is provided in conjunction with a reactor or chip, it may be positioned anywhere on or within the reactor or chip. For example, the light-interacting component may be placed within or adjacent to a reaction site. In some cases, the light-interacting component is integrally connected with the reaction site, for example, as a wall or a surface of the reaction site.

As another example, the light-interacting component may be positioned elsewhere in, or integrally connected to, a chip as disclosed herein, such that at least a portion of light entering the light-interacting component is in optical communication with the reaction site. As used herein, the term “optical communication” generally refers to any pathway that provides for the transport of electromagnetic radiation, such as visible light. Optical communication includes direct, “line-of-sight” communication. Optical communication may also be facilitated, for example, by the use of optical devices such as lenses, filters, optical fiber, waveguides, diffraction gratings, mirrors, beamsplitters, prisms, and the like. In some embodiments, the light-interacting component may direct light to or from more than one reaction site, or the light-interacting component may direct light from more than one light source to a reaction site. In certain embodiments, more than one light-interacting component may be present.

The light-interacting component may include a waveguide in some cases. The term “waveguide,” as used herein, is given its ordinary meaning in the art and may include optical fibers. A waveguide is generally able to receive light and guide or transmit a portion of that light to a destination not within “line-of-sight” communication (although a waveguide can transmit light to a line-of-sight region), e.g., around bends, corners, and similar obstacles without substantial losses.

In some embodiments, a waveguide may include a “core” region of material embedded or surrounded, at least in part, by a second “cladding” material, which may have a lower refractive index. The core may have any shape, for example, a slab, a strip, or a cylinder of material.

The waveguide, or at least a portion of the waveguide, may be fashioned out of any material able to transmit or light to or from the reaction site. The waveguide may be substantially transparent, or translucent in some cases. In some embodiments, the waveguide may be formed out of a silicon-based material, for example, glass, ion-implanted glass, quartz, silicon, silicon oxide, silicon nitride, silicon carbide, polysilicon, coated glass, conductive glass, indium-tin-oxide glass and the like. In other embodiments, the waveguide may comprise other transparent or translucent organic or inorganic materials. For example, in certain embodiments, the waveguide may comprise a polymer including, but not limited to, polyacrylate, polymethacrylate, polycarbonate, polystyrene, polypropylene, polyethylene, polyimide, polyvinylidene fluoride, an ion-exchanged polymer, and fluorinated derivatives of the above. Combinations, blends, or copolymers are also possible.

In certain embodiments, the waveguide or a portion thereof may be surrounded by or coated with a highly reflective material, for example, silver or aluminum. In another embodiment, the waveguide may be fashioned such that it comprises a central material (e.g., a core) having a first index of refraction, and a surrounding material (e.g., a cladding) having a second index of refraction. The cladding may have an index of refraction that is less than the index of refraction of the central material. In yet another embodiment, the index of refraction of the core or the cladding may vary over the cross section. As one example, the core may be a graded index optical fiber, where the index of refraction is generally highest near the center of the core.

Under these conditions, a substantial portion of the light traveling through the central material may be internally reflected (“total internal reflection”) as a result of this refractive index difference. Electromagnetic radiation entering one end of the waveguide may be confined to the central region due to the phenomenon of total internal reflection at the core-cladding boundary. The light may be transported through the core, without significant absorption by the cladding material or other surrounding materials, until it reaches the end of the waveguide, or a predetermined region of the waveguide that light is allowed to exit from. Light traveling through the central material may be directed around corners and other obstacles without a significant loss of intensity, for example, with an attenuation coefficient of less than about 10 db/cm or 20 db/cm. In another embodiment, the waveguide may have more than one central material or more than one surrounding material.

As one example of a waveguide, both the central and surrounding materials forming the waveguide may each be a glass. As another example, a waveguide may be formed out of a polymer and a silicon-based material, such that the material with the lower index of refraction surrounds the material with the higher index of refraction. As yet another example, the waveguide may be constructed out of a single material surrounded by, for example, air or a portion of the chip having a higher index of refraction than the waveguide, thus resulting in a condition where total internal reflection may occur at the waveguide/air or waveguide/chip interface.

The waveguide may be constructed by any suitable technique known to those of ordinary skill in the art, for example, by milling, grinding, or machining (e.g., by cutting or etching a channel into a chip substrate, then depositing material into the channel, optionally using a sealant). The waveguide may also be formed, for example, by depositing layers of materials during the chip fabrication process. The deposited material, in some cases, can have a higher index of refraction than the surrounding substrate, thus forming a general core-cladding structure, as previously described. The waveguide may also be constructed by laser etching of materials forming the chip, such as glass or plastic, in such a way as to manipulate/alter the refractive index, relative to the surrounding material. In some cases, the refractive index of the etched/non-etched portion may be controlled so as to create a core-cladding structure.

In some embodiments, the light-interacting component may be, or include, a source of light. The light source may be any light source in optical communication with the reaction site. For example, the light source may be external or ambient light, a coherent or monochromatic beam of light such as created in an LED, or a laser such as a semiconductor laser or a quantum well laser. The light source may be integrally connected with a portion of the chip, for example, in a laser diode fabricated as part of the chip, or the light source may be separate from the chip and not integrally connected with it, but still positioned so as to allow optical communication with the reaction site. The light source may produce a single wavelength or a substantially monochromatic wavelength, or a wide range of wavelengths, as previously described. The source of light, in certain embodiments, may also be generated in a chemical reaction or a biological process, such as a chemical reaction that produces photons, for example, a reaction involving GFP (“green fluorescence protein”) or luciferase, or through fluorescence or phosphorescence. For example, incident electrons, electrical current, friction, heat, chemical or biological reactions may be applied to generate light, for example, within a sample located within a reaction site, or from a reaction center located within the chip in optical communication with the reaction site.

In certain cases, the light-interacting component may include a filter, for example, a low pass filter, a high pass filter, a notch filter, a spatial filter, a wavelength-selecting filter, or the like. The filter may be able to, for example, substantially reduce or eliminate a portion of the incident light. For example, the filter may eliminate or substantially reduce light having a wavelength below about 350 nm or greater than about 1000 nm. In another embodiment, the filter may be able to reduce noise within the incident light, or increase the signal-to-noise ratio of the incident light. In still another embodiment, the filter may be able to polarize the incident light, for example, linearly or circularly.

In some embodiments, the light-interacting component may include an optical element in optical communication with the reaction site. As used herein, an “optical element” refers to any element or device able to alter the pathway of light entering or exiting the optical element, for example, by focusing or collimating the light, or causing the light to diverge. For example, the optical element may focus the incident light to a single point or a small region, or the optical element may collimate or redirect divergent beams of light to form a parallel or converging beams of light. The term “focus” generally refers to the ability to cause rays of light to converge to a point or a small region. The term “collimate” generally refers to the ability to increase the convergence of rays of light, not necessarily to a point or a small region, for example, such that the beam focuses at an infinite distance. As one example, diverging beams of light may be collimated into parallel beams of light. In certain embodiments, the optical element may disperse or cause light to diverge, for example, as in a diverging lens. In other embodiments, the optical element may be, for example, a beamsplitter, an optical coating (e.g., a dichroic, an antireflective, or a reflective coating), an optical grating, a diffraction grating, or the like.

In one set of embodiments, the optical element may be a lens. The lens may be any lens, such as a converging or a diverging lens. The lens may be, for example, a meniscus, a plano-convex lens, a plano-concave lens, a double convex lens, a double concave lens, a Fresnel lens, a spherical lens, an aspheric lens, a binary lens, or the like. The optical element may also be a mirror, such as a planar mirror, a curved mirror, a parabolic mirror, or the like. In other embodiments, the optical element may cause light to disperse, for example, as in a diffraction grating or a prism.

In certain cases, a material having a different index of refraction may be used. For example, in embodiments in which light reaches the optical element through a waveguide, the optical element may be a material having a different index of refraction than the waveguide. In some cases, the index of refraction of the optical element will be about the same as or more than the index of refraction of the waveguide.

In some cases, a material having a graded index of refraction (a “GRIN” material) may be used as an optical element. The GRIN material may minimize the amount of divergence inherent in light reaching the GRIN material. For example, a material of uniform thickness can be made to act as a lens by varying its refractive index along a cross section of the element. In one embodiment, the GRIN material may redirect divergent rays of light into a parallel arrangement. In another embodiment, the GRIN material does not necessarily have a uniform thickness, and a combination of the graded index of refraction of the material and the shape of the material may be used to focus or collimate the light.

The light-interacting component, in some embodiments, may include a component that is able to convert light to electricity, such as a photosensor or photodetector, a photomultiplier, a photocell, a photodiode such as an avalanche photodiode, a photodiode array, a CCD chip (“charge-coupled device”) or the like. The component may be used, in some cases, to determine the state or condition of a substance within a reaction site, for example, through emission (including fluorescence or phosphorescence), absorbance, scattering, optical density, polarization measurements, or other measurements, including using the human eye.

In certain cases, the light-interacting component may be used for imaging purposes, for example, to image a portion of a cell or other material located at or near the reaction site, or to determine whether a cell has adhered to a surface.

In some cases, the light-interacting component may be used to produce electricity. In one embodiment, a photocell may be integrally fabricated within the chip using one or more layers comprising semiconductor materials.

In some embodiments, light may be directed to the reaction site, for example, to activate or inhibit a chemical reaction. For example, a reaction may require the use of light for activation, or a light-sensitive enzyme may be inhibited by applying light to the enzyme. In certain embodiments, light directed to the reaction site may be used as a probe or a signal source. The light may be delivered in a controlled manner to the reaction site in certain embodiments, for example, so that the light reaching the reaction site has a specific wavelength, polarization, or intensity.

In some embodiments, a portion of the light arising from the reaction site may be detected and analyzed. The light arising from the reaction site may be reflected or refracted light, for example, light directed to the reaction as previously described, or the light may be produced through physical means, for example, through fluorescence or phosphorescence. In certain embodiments, the light may be generated within the reaction site, as previously described. Light from the reaction site may be analyzed using any suitable analytical technique, for example, infrared spectroscopy, FTIR (“Fourier Transform Infrared Spectroscopy”), Raman spectroscopy, absorption spectroscopy, fluorescence spectroscopy, optical density, circular dichroism, light scattering, polarimetry, refractometry, turbidity measurements, quasielectric light scattering, or any other suitable techniques. In another embodiment, imaging of the reaction site may be performed, for example using optical imaging, or infrared imaging.

In some embodiments, a reactor and/or a reaction site within a chip may be constructed and arranged to maintain an environment that promotes the growth of one or more types of living cells, for example, simultaneously. In some cases, the reaction site may be provided with fluid flow, oxygen, nutrient distribution, etc., conditions that are similar to those found in living tissue, for example, tissue that the cells originate from. Thus, the chip may be able to provide conditions that are closer to in vivo than those provided by batch culture systems. In embodiments where one or more cells are used in the reaction site, the cells may be any cell or cell type, for instance a prokaryotic cell or a eukaryotic cell. For example, the cell may be a bacterium or other single-cell organism, a plant cell, an insect cell, a fungi cell or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some cases, the cell may be a genetically engineered cell. In certain embodiments, the cell may be a Chinese hamster ovarian (“CHO”) cell or a 3T3 cell. In some embodiments, more than one cell type may be used simultaneously, for example, fibroblasts and hepatocytes. In certain embodiments, cell monolayers, tissue cultures or cellular constructs (e.g., cells located on a non-living scaffold), and the like may also be used in the reaction site. The precise environmental conditions necessary in the reaction site for a specific cell type or types may be determined by those of ordinary skill in the art.

In some instances, the cells may produce chemical or biological compounds of therapeutic and/or diagnostic interest, for instance, in nanogram, microgram, milligram or gram or higher quantities. For example, the cells may be able to produce products such as monoclonal antibodies, proteins such as recombinant proteins, amino acids, hormones, vitamins, drug or pharmaceuticals, other therapeutic molecules, artificial chemicals, polymers, tracers such as GFP (“green fluorescent protein”) or luciferase, etc. In one set of embodiments, the cells may be used for drug discovery and/or drug developmental purposes. For instance, the cells may be exposed to an agent suspected of interacting with the cells. Non-limiting examples of such agents include a carcinogenic or mutagenic compound, a synthetic compound, a hormone or hormone analog, a vitamin, a tracer, a drug or a pharmaceutical, a virus, a prion, a bacteria, etc. For example, in one embodiment, the invention may be used in automating cell culture to enable high-throughput processing of monoclonal antibodies and/or other compounds of interest. In another embodiment, the disclosed devices and methods may be used to screen cells, cell types, cell growth conditions, or the like, for example, to determine self viability, self production rates, etc. In some cases, the disclosed devices and methods may be used in high through put screening techniques. For example, the disclosed devices and methods may be used to assess the effect of one or more selected compounds on cell growth, normal or abnormal biological function of a cell or cell type, expression of a protein or other agent produced by the cell, or the like. The disclosed devices and methods may also be used to investigate the effects of various environmental factors on cell growth, cell biological function, production of a cell product, etc.

In certain cases, a reactor and/or a reaction site within a chip may be constructed and arranged to prevent, facilitate, and/or determine a chemical or a biochemical reaction with the living cells within the reaction site (for example, to determine the effect, if any, of an agent such as a drug, a hormone, a vitamin, an antibiotic, an enzyme, an antibody, a protein, a carbohydrate, etc. on a living cell). For example, one or more agents suspected of being able to interact with a cell may be added to a reactor and/or a reaction site containing the cell, and the response of the cell to the agent(s) may be determined, using the systems and methods of the invention.

In some cases, the cells may be sensitive to light. For example, the cell may be a plant cell that responds to a light stimulus or is photosynthetic. In another embodiment, the light may be used to grow cells, such as mammalian cells sensitive to light, or plant cells. In yet another embodiment, the cell may be a bacterium that is attracted to or is repelled by light. In another embodiment, the cell may be an animal cell having a light receptor or other light-signaling response, for example, a rod cell or a cone cell. In yet another embodiment, the cell may be a genetically engineered cell having a light receptor or another light-sensitive molecule, for example, one that decomposes or forms reactive entities upon exposure to light, or stimulates a biological process to occur. In other cases, the cell may be insensitive to light; light applied to the chip may be used for analysis of the cells, for example, detection, imaging, counting, morphological analysis, or spectroscopic analysis. In still other cases, the light may be used to kill the cells, for example, directly, or by inducing an apoptotic reaction.

In some embodiments, the chip may be constructed and arranged such that cells within the chip can be maintained in a metabolically active state, for example, such that the cells are able to grow and divide. For instance, the chip may be constructed such that one or more additional surfaces can be added to the reaction site, for example, as in a series of plates, or the chip may be constructed such that the cells are able to divide while remaining attached to a substrate. In some cases, the chip may be constructed such that cells may be harvested or removed from the chip, for example, through an outlet of the chip, or by removal of a surface from the reaction site, optionally without substantially disturbing other cells present within the chip. The chip may be able to maintain the cells in a metabolically active state for any suitable length of time, for example, 1 day, 1 week, 30 days, 60 days, 90 days, 1 year, or indefinitely in some cases.

In certain embodiments, the any of the above-mentioned chips may be packaged in kits, optionally including instructions for use of the chips. That is, the kit can include a description of use of the chip, for example, for use with a microplate, or an apparatus adapted to handle microplates. As used herein, “instructions” can define a component of instruction and/or promotion, and typically involve written instructions on or associated with packaging of the invention. Instructions also can include any oral or electronic instructions provided in any manner such that a user of the chip will clearly recognize that the instructions are to be associated with the chip. Additionally, the kit may include other components depending on the specific application, for example, containers, adapters, syringes, needles, replacement parts, etc. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention.

The following do not exemplify the full scope of the invention, which is defined only by the claims appended below.

### EXAMPLE 1

In this example, an exemplary chip, as illustrated generally in FIG. 4A, is prepared. A first chip layer having associated fluidic channels, ports, compartments, other reaction sites, etc. therein is injection molded or machined from a stock sheet of acrylic or polycarbonate. This first layer is attached to a machined or injection molded flat bottom plate (also acrylic or polycarbonate) by means of a pressure-sensitive silicone adhesive (Dielectric Polymers). A humidity control membrane comprising one or more of the materials described above for forming humidity control layers/membranes is also attached to the top side of the first layer by means of the pressure-sensitive silicone adhesive.

A second chip layer (including a top) having associated fluidic channels, ports, compartments, other reaction sites, etc. therein is cast in a mold using PDMS. This second layer is fashioned to be alignable with the first chip layer. The second layer is aligned with the compartments in the first chip layer and attached by means of the pressure-sensitive silicone adhesive, forming a completed chip. The PDMS top could function as a septum or a self-sealing membrane by itself, or in some cases, an additional partial layer of PDMS could be bonded over an inlet or outlet of the chip using the pressure-sensitive adhesive.

### EXAMPLE 2

This example illustrates various chips formed from multiple layers of dissimilar materials. A variety of adhesives can be used to fix the interface layers to the rigid cell culture or sealing layers depending on the materials involved. One adhesive that can be used for bonding PDMS to polycarbonate is a two-part urethane epoxy mixed with un-cured PDMS. The adhesive process used to bond rigid polycarbonate layers to each other can be either sonic welding or a heated press. The reaction site is designed, in this example, to be about 200 microns thick and had a volume of roughly 20 microliters.

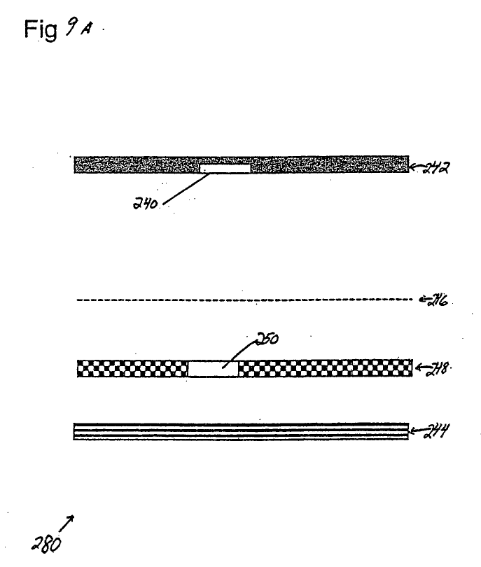

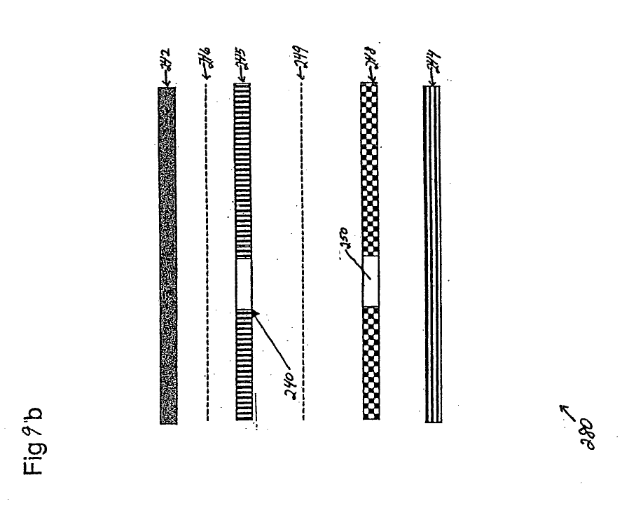

A chip 280 having reaction site 240 is fabricated. As shown in FIG. 8A, a polycarbonate layer 244 is attached to a humidity control material of the invention 242. A gap within the material 242 defines reaction site 240 when the chip was assembled, as shown in FIG. 8A. Material 242 is attached to polycarbonate layer 244.

A similar chip is illustrated in FIG. 8B. In this figure, reaction site 240 is defined by layer 245, which is a thin, rigid layer of polycarbonate. Between layers 242 and 245 is a humidity control material 246, comprising one or more of the materials described above for forming humidity control layers/membranes. Layers 244, 245, 246 and 242 of chip 80 are joined using the above-described adhesive processes.

### EXAMPLE 3

This example illustrates various chips formed from multiple layers of dissimilar materials. A variety of adhesives can be used to fix the interface layers to the rigid cell culture or sealing layers depending on the materials involved. One example adhesive used for bonding PDMS to polycarbonate is a two-part urethane epoxy mixed with un-cured PDMS. The adhesive process used to bond rigid polycarbonate layers to each other can be, for example, sonic welding or a heated press. The reaction site is designed, in this example, to be about 200 microns thick and had a volume of roughly 20 microliters.

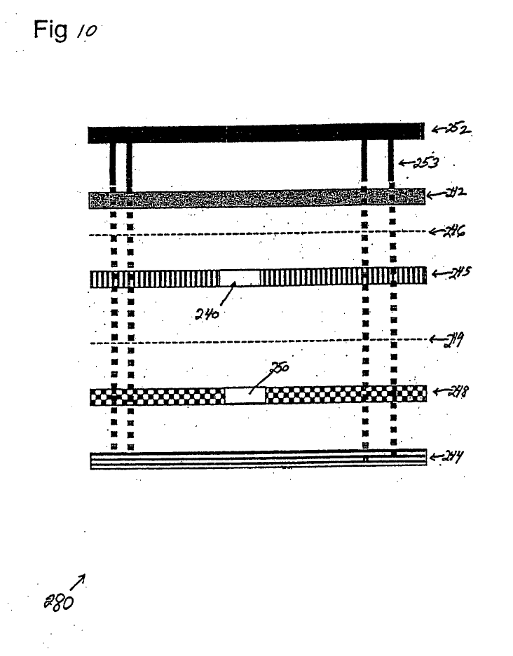

The fabrication of the chips illustrated in FIGS. 9A and 9B is similar to those described in Example 2, including the adhesion methods. In FIG. 9A, the reservoir layer 248 is fashioned from polycarbonate and was positioned between a humidity control material of the invention 246 and polycarbonate layer 244. Reservoir layer 248 has a gap (i.e., a hole or a partially hollowed out space) that defines reaction site 50, which was a reservoir in this example. In FIG. 9A, the reaction site 240 is defined by a gap interface layer 242.

In FIG. 9B, polycarbonate layer 248 is used to define reaction site 250. Additionally, a humidity control membrane 249 comprising one or more of the materials described above for forming humidity control layers/membranes is used between polycarbonate layer 245 (defining reaction site 240) and polycarbonate layer 248.

### EXAMPLE 4

In this example, a chip sealed by a membrane is fabricated, which membrane may be formed of one or more of the materials described above for forming humidity control layers/membranes, having a permeability to oxygen high enough to allow culture of living cells. The amount of oxygen required in this example is a function of the number of cells present and the oxygen requirements for the cells' metabolism. This is illustrated in the Equations 1-3 below:

$$\begin{matrix}V = Ad & ( 1 ) \\ P = \frac{nrdl}{{p}_{in} - {p}_{out}} & ( 2 ) \\ \frac{PA ( {p}_{in} - {p}_{out} )}{l} = \frac{Δ {m}_{gas}}{Δ t} = nrV & ( 3 )\end{matrix}$$

In these equations, P represents the permeability (typically measured in units of cm³_{STP} mm/m² atm day), A is the area (typically measured in m²), pᵢₙ is the oxygen partial pressure in the chip (typically measured in atm), pₒᵤₜ is the oxygen partial pressure outside the chip (typically measured in atm), l is the membrane thickness (typically measured in micrometers), V is the volume of the chip (typically measured in microliters), d is the cell culture compartment depth (typically measured in micrometers), n is the cell density (typically measured in cell/ml), and r is the specific oxygen demand per cell (typically measured in O₂/cell h).

Equation 3 represents a mass balance equating oxygen consumed by the growing culture to that available via diffusion through the film. Equation 1 sets the volume of the cell culture compartment equal to the cross sectional area of the membrane contacting the compartment. Rearrangement yields Equation 2, thus expressing the minimum oxygen permeability needed to sustain cells of a given population density and metabolic rate as a function of film thickness and compartment depth.

Values for P generally depend on the polymer and the permeant system, and were varied in this example for oxygen between 39000 (cm³_{STP} mm/m ² atm day) for silicon to 0.01 (cm³_{STP} mm/m² atm day) for ethylene vinyl acetate (“EVA”); pᵢₙ was varied between 0.05 atm and 0.2 atm, and pₒᵤₜ was assumed to be 0.2 atm. The film thickness, l, was varied between 1 micrometer and 2 mm. V was held to be less than 1 ml, and the cell culture depth, d, ranged between 30 micrometers and 2 mm. The cell density, n, was assumed in this example to be between 10⁵ cells/ml and 10⁷ cells/ml for mammalian cells and between 10⁹ cells/ml and 10¹¹ cells/ml for bacteria. The specific oxygen demand per cell ranged between 0.5×10⁻¹² and 5×10⁻¹² mol O₂/cell h.

### EXAMPLE 5

The example illustrates a calculation of kₗa and specific exchange coefficients useful in the design and fabrication of a chip sealed by a membrane, in accordance with one embodiment of the invention. One equation often used to describe mass transfer of oxygen into the liquid phase of a bioreactor is (4) below:

$$\frac{{dm}_{gas}}{dt} = {k}_{1} a ( {C}^{*} - C ) V \qquad ( 4 )$$

where dm/dt is the mass flux of oxygen, and kₗ is the transfer coefficient at the phase boundary, with transfer being a mixture of convection and diffusion. In the equation, a is the specific exchange surface, in units of m⁻¹, and may be interpreted physically as the total surface are of gas exchanging bubbles per volume of liquid. The concentration driving force is represented by (C*−C), with C* representing the equilibrium gas concentration on the liquid side of the phase boundary, and C representing the bulk concentration of the gas phase species in the liquid. The volume of the liquid phase is represented by V.

The oxygen flux is determined by diffusion through the walls of the cell culture chamber as shown in Equation 5:

$$\frac{{dm}_{gas}}{dt} = \frac{A Π ( {P}^{*} - P )}{l} \qquad ( 5 )$$

where A is the surface area of the phase boundary for gas exchange, Π is the permeability of the membrane in g mm/(m² atm day), and (P*−P) is the concentration driving force in atmospheres, expressed as partial pressure P on the downstream or wet side of the membrane relative to partial pressure, P* on the upstream or dry side of the membrane.

Application of Henry's law for gas solubility (Equation 6) with H as the Henry's law constant relates C* to P*, substituting in Equation 5 and setting Equations 4 and 5 equal, followed dividing both equations by A, the vessel cross-sectional area, yields Equation 7:

$$\begin{matrix}{C}^{*} = \frac{{P}^{*}}{H} & ( 6 ) \\ {k}_{1} a ( {C}^{*} - C ) d = \frac{Π ( {C}^{*} - C ) H}{l} & ( 7 )\end{matrix}$$

where d is the vessel depth and the P and P* terms have been replaced by their Henry's law relationships. Dividing both sides by (C*−C) cancels the concentration terms making Kₗa a function of materials properties and geometry only:

$${k}_{l} a = \frac{H Π}{ld} \qquad ( 8 )$$

For a given value of H, which may be dictated by temperature, Kₗa can be made larger or smaller by changing membrane thickness l, compartment depth d, or membrane materials and thus the permeability.

As a specific, non-limiting example, evaluating Henrys law constant for 37° C., using polydimethylsiloxane:

$$H = ( \frac{0.21 atm}{8.5 mg \text{/} liter} ) ( \frac{32000 mg}{mol} ) = 790.6 \frac{atm liter}{mol} \qquad ( 9 )$$

Substituting the PDMS permeability of 39000 (cm³_{STP} mm/m² atm day) and expressing compartment depth and film thickness in millimeters yields:

$${K}_{l} a = ( 790.6 \frac{atm liter}{mol} ) ( 39000 \frac{{cm}_{STP}^{3} mm}{{m}^{2} atm day} ) ( \frac{1}{mm} ) ( \frac{1}{mm} ) \qquad ( 10 )$$

Converting cm³_{STP} to moles:

$${K}_{l} a = ( 790.6 \frac{atm liter}{mol} ) ( 39000 \frac{{cm}_{STP}^{3} mm}{{m}^{2} atm day} ) ( \frac{1 mol}{\text{22,400} {cm}_{STP}^{3}} ) ( \frac{1}{mm} ) ( \frac{1}{mm} ) \qquad ( 11 )$$

Thus:

$${k}_{l} a = ( 790.6 ) ( 39000 ) ( \frac{1}{22400} ) ( \frac{1}{mm} ) ( \frac{1}{mm} ) ( \frac{1}{24 hr} ) \qquad ( 12 )$$

Therefore:
Kₗa=(53.2/ld) with l and d in millimeters for the PDMS system of this example.

### EXAMPLE 6

In this example, the calculation and control of dissolved oxygen levels in a chip having a reaction site, according to one embodiment, is demonstrated. An oxygen balance on the liquid phase of a bioreactor is shown as Equation 13 below.

$$\frac{d ( V [ C ] )}{dt} = \frac{A Π ( {P}^{*} - P )}{l} - ρ RV \qquad ( 13 )$$

Taking the reaction site volume, V, as a constant, the rate of change of oxygen concentration, C, in time is a function only of the supply of oxygen available via diffusion through the reaction site walls (the first term on the right hand side of the equation) and the uptake of oxygen by the living cells (the rightmost term of the equation). The diffusive flux through the reaction site walls is proportional to A, the area available for exchange, the permeability of the film material, Π, and the concentration driving force of oxygen across the film, (P*−P) where P* is the external oxygen partial pressure, and P is the partial pressure on the downstream, or wet side of the film. The permeability is inversely proportional to the membrane thickness, l. Oxygen uptake is expressed as the product of the cell population density, ρ, (cells/volume) and the specific cellular consumption rate, R and the reaction site volume, V.

If the oxygen level is to be maintained at a stable value, the system should reach a steady state where oxygen uptake rate balances oxygen flux arriving via diffusion through the reaction site walls. That is, the derivative is set to 0. Furthermore, inspection of Equation 13 shows that A, Π, l, and V are all parameters of materials or geometry of chip construction and thus may be treated as constants in this example. Thus, according to this example, R, the specific cellular oxygen uptake rate and ρ, the cell copulation are the only time dependent parameters that influence the difference between P* and P. Modeled as a control scheme, P may be treated as a controlled variable, P* as a manipulated variable, and the relationship between them, or gain, is unity with an offset mostly dictated by a set of constant parameters A, P, l, V, and the weakly time dependent terms R and ρ.

Rearranging Equation 13 to highlight this relationship gives Equation 14 below, after noting that the exchange area A is twice the cross-sectional area of the reaction site of this example. Normalizing by P* by P^{atm} allows expression of oxygen levels as a fraction of atmospheric pressure, shown in Equation 15:

$$\begin{matrix}P = P * \frac{ρ Rdl}{2 Π} & ( 14 ) \\ \% DO = DO * - \frac{ρ Rdl}{2 Π {P}_{O2}} & ( 15 )\end{matrix}$$

where d is the reaction site depth, resulting from dividing reaction site volume by cross-sectional area.

Table 1 below presents % DO as a function of cell density for several chip designs (values of dl). R is assumed to be a typical value of the specific oxygen consumption rate, 0.5 pmol/cell/hr in this example. As an order-of-magnitude estimate in this example, DO inside the cell culture reaction site is 5.9% below the external set point for each million cells per ml in the culture reaction site.

TABLE 1

| Cells/ml | dl (mm²) | Offset (% atm) | Offset (% O₂) |
|---|---|---|---|
| 1 × 10⁶ | 0.25 | 1.25% | 5.9% |
| 1 × 10⁶ | 0.125 | 0.65% | 3.0% |
| 1 × 10⁷ | 0.25 | 12.5% | 59% |
| 1 × 10⁷ | 0.125 | 6.25% | 29.5% |

The same analysis may be applied to levels of carbon dioxide generated in the reaction site by the growing cells, accounting for the fact that the buffer system may tend to include bicarbonate, which can participate by absorbing or releasing CO₂.

The following is an example of the calculation used to construct Table 1:

$$\frac{dl}{2 Π {P}_{O2}^{atm}} = \frac{0.25 {mm}^{2}}{\begin{matrix}2 ( 2500 \frac{{cm}_{STP}^{3} mm}{{m}^{2} atm day} ) ( \frac{1 mol}{22400 {cm}_{STP}^{3}} ) \\ {( \frac{1 m}{1000 mm} )}^{2} ( \frac{1 day}{24 hr} ) ( 1 atm )\end{matrix}} = 2.5 \times {10}^{7} \frac{hr {mm}^{3}}{mol} \qquad ( 16 )$$

A baseline value for ρ is 10³ cells/mm³ and for R is 0.5×10⁻¹² mol/cell/hr. Thus:

$$\begin{matrix}DO * - DO = 2.5 \times {10}^{7} ( \frac{hr {mm}^{3}}{mol} ) 5 \times {10}^{- 13} ( \frac{ mol}{cell hr} ) ρ \frac{cells}{{mm}^{3}} \\ or \text{:} & ( 17 ) \\ \% DO * - \% DO = 1.25 \times {10}^{- 5} ( \frac{{mm}^{3}}{cell} ) ρ \frac{cells}{{mm}^{3}} 100 \% & ( 18 )\end{matrix}$$

Literature values used for the oxygen uptake are: 0.05 to 5 pmol/cell/hour, 0.4 pmol/cell/hour, 0.3 pmol/cell/hour, 0.018 to 0.036 pmol/hr cell, and 0.1 pmol/cell/hr.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

In the claims, as well as in the specification above, all transitional phrases such as “comprising,” “including,” “carrying,” “having,” “containing” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An apparatus, comprising:
a substrate comprising a predetermined reaction site having a volume of less than about 2 ml; and
a material positioned adjacent the predetermined reaction site, the material comprising a polymer including a structure: 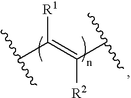
wherein n is at least 1, and each of R¹ and R² independently comprises an atom, such that, simultaneously, R¹ is not methyl and R² is not trimethylsilyl.

2. An apparatus as in claim 1, wherein the predetermined reaction site has a volume of less than about 1 ml.

3. An apparatus as in claim 1, wherein the substrate comprises at least 2 predetermined reaction sites.

4. An apparatus as in claim 1, wherein the at least 2 predetermined reaction sites are not in fluid communication with each other.

5. (canceled)

6. An apparatus as in claim 1, wherein the substrate is constructed and arranged to maintain at least one living cell at the predetermined reaction site.

7-13. (canceled)

14. An apparatus as in claim 1, wherein R¹ is an alkyl.

15. (canceled)

16. An apparatus as in claim 1, wherein R² is an alkyl.

17-25. (canceled)

26. An apparatus as in claim 1, further comprising a gas head space in gaseous communication with the predetermined reaction site.

27. An apparatus as in claim 26, wherein the gas head space is separated from the predetermined reaction site by a membrane.

28. An apparatus as in claim 1, further comprising an inlet port, an outlet port, and at least one microfluidic channel, each in fluid communication with the predetermined reaction site.

29. An apparatus as in claim 28, further comprising a self-sealing elastomeric material defining portions of the inlet and outlet ports.

30. An apparatus as in claim 28, wherein the inlet and outlet ports are both accessible from a first side of the substrate.

31. (canceled)

32. An apparatus as in claim 31, wherein an adhesive layer binds the material to the substrate.

33. (canceled)

34. An apparatus, comprising:
a substrate comprising a predetermined reaction site having a volume of less than about 2 ml; and
a material positioned adjacent the predetermined reaction site, the material comprising a polymer including a structure: 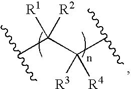
wherein n is at least 1, and each of R¹, R², R³, and R⁴ independently comprises an atom such that, simultaneously, R¹ is not H, R² is not H. R³ is not H, and R⁴ is neither H nor has a structure: 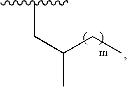
m being an integer between 0 and 3 inclusive.

35-57. (canceled)

58. A method, comprising an act of:
culturing at least one living cell in a predetermined reaction site having a volume of less than about 2 ml, the cell proximate a material comprising a polymer including a structure: 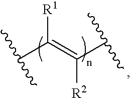
wherein n is at least 1, and each of R¹ and R² independently comprises an atom, such that, simultaneously, R¹ is not methyl and R² is not trimethylsilyl.

59-80. (canceled)

81. A method, comprising an act of:
culturing at least one living cell in a predetermined reaction site having a volume of less than about 2 ml, the cell proximate a material comprising a polymer including a structure: 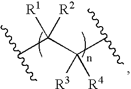
wherein n is at least 1, and each of R¹, R², R³, and R⁴ independently comprises an atom such that, simultaneously, R¹ is not H, R² is not H, R³ is not H, and R⁴ is neither H nor has a structure: 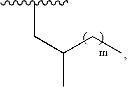
m being an integer between 0 and 3 inclusive.

82-103. (canceled)

104. An apparatus, comprising:
a substrate comprising a predetermined reaction site having a volume of less than about 2 ml; and
a material positioned adjacent the predetermined reaction site, the material comprising a polymer including a structure: 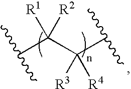
wherein n is at least 1, and each of R¹, R², R³, and R⁴ independently is hydrogen, a halogen, or a carbon-containing moiety, such that at least one of R¹, R², R³, or R⁴ is a carbon-containing moiety that comprises a halogen.

105-129. (canceled)

130. A method, comprising an act of:
culturing at least one living cell in a predetermined reaction site having a volume of less than about 2 ml, the cell proximate a material comprising a polymer including a structure: 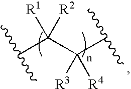
wherein n is at least 1, and each of R¹, R², R³, and R⁴ independently is hydrogen, a halogen, or a carbon-containing moiety, such that at least one of R¹, R², R³, or R⁴ is a carbon-containing moiety that comprises a halogen.

131-154. (canceled)

155. An apparatus, comprising:
a substrate comprising a predetermined reaction site having a volume of less than about 2 ml; and
a material positioned adjacent the predetermined reaction site, the material comprising a polymer including a structure: 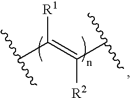
wherein n is at least 1, and each of R¹ and R² independently comprises an atom such that at least one of R¹ or R² is a carbon-containing moiety that comprises a halogen.

156-175. (canceled)

176. A method, comprising an act of:
culturing at least one living cell in a predetermined reaction site having a volume of less than about 2 ml, the cell proximate a material comprising a polymer including a structure: 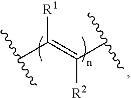
wherein n is at least 1, and each of R¹ and R² independently comprises an atom such that at least one of R¹ or R² is a carbon-containing moiety that comprises a halogen.

177-198. (canceled)
